(19) United States
(12) Patent Application Publication
(10) Pub. No.: US 2005/0095588 A1
(43) Pub. Date: May 5, 2005

(54) Process for identifying membrane protein drug targets

(76) Inventors: Kai Wang, Bellevue, WA (US); Katherine Hsiao-Wen Huang, Seattle, WA (US); John Jay Liljeberg, Mill Creek, WA (US)

Correspondence Address: Thomas J. Bordner;MEDLEN & CARROLL, LLP, 101 Howard Street, Suite 350, San Francisco, CA, 94105, US

(21) Appl. No.: 10/282,549

(22) Filed: Oct. 29, 2002

Related U.S. Application Data
(60) Provisional application No. 60/349,220, filed on Oct. 29, 2001.

(51) Int. Cl. C12Q 1/68

(52) U.S. Cl. 435/6

(57) ABSTRACT

The present invention relates to a streamlined method to develop antibody therapeutics based on the identification of differentially expressed genes, and in particular, the present invention relates to systems and methods for differentiating between the expression of genes encoding cell surface proteins in two or more biological samples.

This application claims priority to U.S. Provisional Application Ser. No. 60/349,220 filed Oct. 29, 2001. The aforementioned Provisional Application is specifically incorporated herein by reference in its entirety.

### FIELD OF THE INVENTION

The present invention relates to a streamlined method to develop antibody therapeutics based on the identification of differentially expressed genes, and in particular, the present invention relates to systems and methods for differentiating between the expression of genes encoding cell surface proteins in two or more biological samples.

### BACKGROUND OF THE INVENTION

In higher organisms, any given cell expresses only a fraction of the total number of genes present in its genome. The small fraction of expressed genes determine the life processes carried out by the cell, e.g., development and differentiation, homeostasis, response to insults, cell cycle regulation, aging, apoptosis, and the like. Alterations in gene expression decide the course of normal cell development and often the appearance of diseased states (e.g., cancer). Many disease states are characterized by differences in the expression levels of various genes either through changes in the copy number of the DNA or through changes in transcription levels of particular genes (e.g., through control of initiation, provision of RNA precursors, RNA processing, etc.).

In particular, losses and gains of genetic material play an important role in malignant transformation and progression (i.e., tumorgenesis). These gains and losses are thought to be “driven” by at least two kinds of genes. Oncogenes are positive regulators of tumorgenesis, while tumor suppressor genes are negative regulators of tumorgenesis. Various mechanisms of activating unregulated growth include increasing the number of oncogenes that encode oncogenic proteins or to increase the expression levels of these genes. Alternatively, the loss of genetic material or a decrease in the level of expression of genes that code for tumor suppressors can also promote tumorgenesis. Changes in the expression (transcription) levels of particular genes (e.g., oncogenes or tumor suppressors) serve as signposts for the presence and progression of various cancers.

Viral infections are often characterized by elevated expression levels of the viral genes. For example, outbreaks of Herpes simplex, Epstein-Barr virus (e.g., infectious mononucleosis), cytomegalovirus, Varicella-zoster virus infections, parvovirus infections, human papillomavirus infections HTLV, BLV, etc. are all characterized by elevated expression of viral genes. Detecting elevated expression levels of characteristic viral genes provides an effective diagnostic of the disease state. Detecting expression levels of characteristic viral genes indicates active proliferating (and presumably infective) viral disease states.

Consequently, identification of differentially-expressed genes can also provide a key to the diagnosis, prognosis, and treatment of a variety of diseases and conditions in animals, including humans, and plants. Many of these methods can also be used to identify the differential expression of genes sequences associated with predisposition to disease, influence of external treatments, and exposure to infectious agents. The identification of differentially expressed genes can aid in the development of new drugs and diagnostic methods for treating or preventing many diseases.

Traditional approaches to identifying antibody targets for diseases involve isolating RNA from a pathological tissue, such as a cancer, and from a normal tissue and then performing various differential gene expression analysis experiments, such as, differential display, subtractive library screening, differential hybridization, and microarray hybridization to identify differentially expressed genes. Various computational tools are then be used to identify potential cell surface proteins from the numerous experimental results. These processes are laborious and unreliable, especially when most of these experiments only generate partial gene sequence or gene fragments.

Differential gene expression analysis is one technique of analyzing gene expression in two or more cells (or tissue types). Differential gene expression analysis allows discrepancies in expression levels between cells to be identified. Gene expression discrepancies can indicate the presence of a disease state in one of the cells (e.g., cancer) or of pathogen such as an invading virus (e.g., presence of Rex protein in cells infected by the HTLV virus).

Differential gene expression analysis begins with researcher or clinician selecting two or more specimens (e.g., tissues) that are suspected of having gene expression discrepancies of interest (e.g., tissues or organs of a healthy state versus diseased state). Next, tagged cDNA, such as by using radiolabeled nucleotides, is generated from the RNA of the respective specimens. The tagged cDNA targets are then used in assays such as hybridization experiments against various selected gene sequences. Differences between the resulting hybridization patterns as well as intensities are detected and related to discrepancies in gene expression between the specimens. Modifications have been made to the basic methods of differential gene expression analysis in order to improved results. One modification was replacing the traditional radioactive labeling of the target nucleic acid sequences with nonisotopic labels, mainly fluorescent labels. Other modifications have focused on improving methods of immobilizing arrays of the gene probes to the surfaces of a variety of solid supports. (See e.g., Guo, Z, et al., Nucleic Acids Res, 22(24):5456-65 [1994]; Pietu, G., et al., Genome Res., 6(6):492-503 [1996]).

Despite advances in differential gene expression profiling technology, many difficulties to their effective use remain. For instance, competitive hybridization between distinct target sequences, nonspecific binding between “target” and “probe,” and formation of secondary structures in target sequences all can produce poor quality results. Current gene expression profiling employs a random approach to selecting cDNA targets further exacerbating these problems. Each of the problems can further contribute to unwanted noise or background signals in the profiling system that undermines the accuracy and performance of gene expression profiling systems.

What is needed are improved systems and methods of distinguishing gene expression levels in two or more biological samples that limit or reduce the amount of noise in the system and that are less laborious to employ.

### SUMMARY OF THE INVENTION

The present invention relates to the development of antibody drugs based on the identification of expressed genes, and in particular, to systems and methods for differentiating between the expression of genes encoding membrane proteins in two or more biological samples using differential gene expression analysis techniques.

The present invention contemplates detecting differential expression of genes in various cell types or disease stages. The present invention employs a preselection step. In one embodiment of the preselection step, one or more sequence databases are screened for target genes before the differential gene expression analysis is applied. The preselection step picks genes (or fragments) that share a characteristic such as domains or motifs. In a preferred embodiment, such preselected genes share the characteristic of having hydrophobic domains associated with membrane proteins (although each gene need not share the same protein (or nucleotide) sequence or number of hydrophobic domains). In a preferred embodiment, nucleic acids encoding transmembrane proteins are preselected (these proteins typically comprise both hydrophobic domains and hydrophilic domains).

In one embodiment, the present invention contemplates a method comprising: a) providing: i) RNA from two or more distinct biological samples, ii) a plurality of nucleic acid sequences immobilized on a solid support, wherein each of said sequences are derived from genes comprise at least one region encoding a hydrophobic domain (or a motif) associated with membrane bound or transmembrane proteins; b) contacting said immobilized sequences with cDNA derived from RNA under conditions such that there is hybridization of at least a portion of said cDNA with at least a portion of said immobilized sequences; and c) detecting said hybridization. While it is not intended that the present invention be limited to the type of RNA used, it is preferred that either total RNA or mRNA be employed in the above-described embodiment. Accordingly, in some embodiments, RNA from two or more distinct biological samples is selected from the group consisting of total RNA and total mRNA. While it is not intended that the present invention be limited by how the immobilized sequences are obtained (or be limited by the means by which their shared characteristic is identified), in preferred embodiments, these sequences are DNA and they are preselected from databases by electronically (or visually) searching for motifs and/or shared domains. Accordingly, in some embodiments, the plurality of nucleic acid sequences comprises DNA. In some of these embodiments, the DNA comprises cDNA. In still other embodiments, the nucleic acid sequences are selected from a genomic database of nucleic acid sequences.

In preferred embodiments, the nucleic acid sequences encode membrane proteins. In some of these embodiments, the membrane proteins comprise one or more transmembrane domains.

In some embodiments, the two or more distinct biological samples comprise eukaryotic cells. In certain other embodiments, the eukaryotic cells comprise mammalian cells. In still further embodiments the mammalian cells comprise human cells. In other embodiments, the human cells are selected from the group consisting of diseased cells and non-diseased cells. In some of these embodiments, the diseased cells are cancer cells. In yet other embodiments, the human cells differentially express one or more mRNA of interest. Additional embodiments contemplate method wherein the one or more mRNA of interest comprises a viral mRNA.

In some embodiments, the two or more distinct biological samples comprise prokaryotic cells. In some of these embodiments, the prokaryotic cells are selected from the group consisting of pathogenic bacterial cells and non-pathogenic bacterial cells.

In a preferred embodiment, said immobilized gene probe sequences are arrayed, i.e., a particular sequence occupies a separate space on the solid support (e.g., a membrane). Such arraying (e.g., by spotting each plurality of homogenous sequences at a predetermined point on a membrane, such as in a grid or x/y relationship, so that a given sequence is known to occupy a given coordinate) permits rapid screening and identification. Importantly, the conditions for hybridization can be low stringency, medium stringency, or high stringency, depending on the number of “hits” one desires. Of course, medium to high stringency conditions are preferred, in order to favor true positives (i.e., hybridization which reflects the existence of a corresponding expressed sequence) and avoid false positives (i.e., hybridization which does not reflect detection of a corresponding expressed sequence) caused by the hybridization of only partially complementary sequences.

As noted above, two or more distinct biological samples can be employed. Using two samples (first second sample) as a convenient example, the method can further comprise the step of d) distinguishing between i) hybridization of labeled cDNA samples derived from both said first and second RNA samples to a particular (i.e., the same) immobilized sequence and ii) hybridization of labeled cDNA samples derived from either said first and second RNA samples (but not both) to a particular immobilized sequence, so as to identify differentially-expressed genes. Again, this is conveniently done with an arrayed set of immobilized probe sequences. For example, the same arrayed set of sequences can be immobilized on two solid supports (e.g., first and second membranes comprising identically arrayed immobilized sequences) and the cDNA derived from said RNA from first sample can be reacted with sequences of said first array, while the cDNA from the second sample can be reacted with the sequences on said second array (both reactions being performed under identical conditions, and therefore identical levels of stringency). Where the first sample represents cancerous cells and the second sample represents normal control cells, the fact that labeled cDNA from the first sample displays hybridization with a particular immobilized probe (which does not hybridize with the labeled cDNA from the second sample) suggest that the particular immobilized probe reflects a differentially expressed gene associated with cancer.

In still further embodiments, the present invention further comprises e) generating polypeptides corresponding to at least a portion of said differentially-expressed genes. In yet another embodiment, the present invention further comprises step f) comprising the step of generating antibodies (or other antigen binding proteins) to said polypeptide fragments.

In yet still further embodiments, the present invention further comprises step g) comprising the steps of: i) contacting said two or more biological samples with said antibodies, and ii) detecting the extent of binding of said antibodies to said two or more biological samples. The ability to generate antibodies that will react with the initial sample (e.g., the sample used to make RNA) or something equivalent to the initial sample (e.g., cells or tissues of the same type or origin), is premised on the notion that the preselected class of molecules, e.g., membrane-bound proteins or transmembrane proteins, react with antibodies, since at least a potion of the proteins are exposed extracellularly. Generating and testing antibodies on the biological sample (whether cells or tissue) allows confirmation that i) the molecule is a cell-surface molecule, and ii) the gene is differentially-expressed (e.g., because the antibody detects the gene product on the first sample but not on the second sample). It is preferred that, in order to carry out steps g) and h) one should preserve at least a portion of said first and second samples (as well as third, fourth, fifth, etc. samples, where a plurality of samples are used) before making the RNA from such samples (i.e., the RNA specified in i) of step a) above). For example, if the sample is a biopsy, a first portion can be used to prepare RNA and a second portion can be used for antibody studies. Of course, where an equivalent sample can be used, such preservation may not be needed (e.g., where the sample is from cell culture, another sample from the same cell culture may be deemed equivalent to the said first sample from the culture).

The present invention is not limited by the nature of biological samples being used. As used herein, the terms “biological sample” and “biological specimen” are used in the broadest sense. In one sense, it can refer to tissues and cells, including cell fragments. In another sense, it is meant to include a specimen or culture (as well as any devices used for obtaining specimens and cultures) obtained from any biological source containing nucleic acids (e.g., plants and microorganism, including viruses). Biological samples may be obtained from animals (including humans) and encompass fluids, solids, and tissues. Biological samples include, but are not limited to blood products, such as plasma, serum and the like, bodily fluids such as urine, fecal matter, cerebrospinal fluid (CSF), semen, and saliva. Biological samples may be obtained from any normal fluid, solid, or tissue, or from an diseased fluid, solid, or tissue. Similarly, the terms “environmental sample” and “environmental specimens” encompass any solid, or fluid taken from the environment that contains nucleic acids. These examples are not to be construed as limiting the sample types applicable to the present invention.

The present invention is not limited by the particular purpose for carrying out the methods described herein. In one medical diagnostic application, it may be desirable to differentiate between normal and cancerous tissue. In one embodiment, the present invention may be used to differentiate between cancer tissue that is metastatic and cancer tissue that is non-metastatic. In yet another embodiment, the present invention may be used to detect drug resistance.

In another medical diagnostic application, it may be desirable to simply detect the presence or absence of specific pathogens (or pathogenic variants) in a clinical sample. In yet another application, it may be desirable to distinguish one species or strain of pathogen from another.

With regard to distinguishing species of microorganisms, the present invention contemplates comparing the expressed genes of two samples suspected to be different species. In another embodiment, a species that is suspected to have changed or diverged from the parent species is compared with the parent species. For example, a species or strain of bacteria may develop different susceptibilities, or resistances, to a drug (e.g., antibiotics) as compared to the parent species; rapid identification of the specific species or subspecies aids diagnosis and allows initiation of appropriate treatment.

In still another medical diagnostic application, it may be desirable to determine the differential expression of one or more viral genes. The present invention contemplates comparing the expression of viral genes (e.g., HTLV rex gene, HIV gag, pol, and env, etc.) in biological samples (e.g., cells) as an indication of viral infection.

The present invention is not limited by the detection means being employed. In this regard, the present invention contemplates using various fluorescent molecules, affinity labels, radioisotopes, and the like. For example, RNA from the biological samples (the targets) can be labeled prior to hybridization with the immobilized sequences on a support. After washing, specifically hybridized labeled target sequences will be detectably bound to the support. In another embodiment, the RNA (or corresponding cDNA) is electrophoresed, transferred to a membrane and contacted with the labeled preselected sequences (which are not immobilized).

The present invention specifically contemplates comparing expression levels in clinical samples. For example, in some embodiments, the methods of the present invention provide a means to detect the expression of genes that indicate a disease state in the tissues or cells comprising the sample. Human cancer cells are specifically contemplated. In some of these embodiments, the clinical samples comprise tissues from any of the internal tissues (e.g., organs, connective tissues, bones, brain, blood cells, immune system cells, etc.) or the skin of animals. In preferred embodiments the animal are mammals. In particularly preferred embodiments, the mammals are humans.

The present invention also contemplates systems and computer programs for designing unique oligonucleotide sets to represent the sequences entered by a user.

In preferred embodiments, the present system comprises: a) providing; 1) sequences for one or more nucleic acids molecules; 2) an information input device, wherein said information input device is capable of receiving information comprising said sequences for one or more nucleic molecules; 3) a memory device; 4) a processor, wherein said processor is configured to generate a set of oligonucleotide sequences that hybridize to said one or more nucleic acid molecules; and 5) an information output device; and b) entering said one or more nucleic acid sequences into said processor; c) processing said sequences for one or more nucleic acids molecules; d) writing said oligonucleotide sequences to said memory device; and e) transferring said oligonucleotide sequences to said information output device.

In another preferred embodiment, the present system comprises: a) providing: 1) an algorithm; 2) a memory device; 3) a processor; 4) an information input device, wherein said information input device receives information on one or more nucleic acid sequences; and 5) an information output device; and b) storing said algorithm in said memory device; c) running said algorithm stored in said memory device on said processor, wherein said processor is configured to generate a set of oligonucleotide sequences that hybridize to said one or more nucleic acid sequences; and c) output of said generated set of oligonucleotide sequences to said information output device.

The systems and programs of the present invention are not limited by the selection of processors, memory storage devices, information input devices, and information output devices. Indeed, the present invention contemplates that processors includes personal computers, mainframe computers, and the like. Likewise, memory storage devices include all forms of optical and magnetic storage media (RAM, ROM, CDROMs, DVDs, magnetic tapes, disks, and cassettes, and the like). The systems and programs of the present invention contemplate a number of information input and output devices, including keyboards, computer mouse, voice recognition systems, and the like. Similarly, the information output devices of the present invention include, but are not limited to, computer monitors, printout devices, and electronic connections to other processors, oligonucleotide synthesis machines, and the like.

The present invention contemplates optimizing the systems and programs of the present invention to run on a variety of operating systems (e.g., Macintosh, OS x, Microsoft, Windows xp, Windows ME, Windows NT, etc., Linux, Unix, and the like).

In some embodiments of the present invention the systems and programs are optimized to send and receiver information to one or more other processors over digital or analog communications networks.

In still some other embodiments, the systems and methods of the present invention are used configured to generate nucleic acid primer sequences (oligonucleotides) that are suitable for use in a range of sequencing, amplification, and detection methods (e.g., PCR). In preferred embodiments, systems and methods of the present invention configured to generate oligonucleotides primer pairs are substantially similar to those embodiments configured to generate oligonucleotides probe candidates. In particularly preferred embodiments, the systems and methods of the present invention are configured to generate either or both oligonucleotides probe candidates and oligonucleotides primer pairs in a single session.

### DESCRIPTION OF THE FIGURES

FIG. 1 shows a comparison of hybridization sensitivity following the use of one embodiment of the method of the present invention.

FIG. 2 shows one embodiment of a Web based graphical user interface configured for receiving user set parameters.

FIG. 3 shows one embodiment of a Web based graphical user interface displayed by the system prior to displaying system output.

FIG. 4 shows various embodiments of Web based graphical user interfaces showing system error.

FIG. 5 shows one embodiment of a Web based graphical user interface showing system output.

FIG. 6 shows one embodiment of a graphical display of the primary set of oligonucleotides generated by the system for a particular nucleic acid sequence.

FIG. 7 shows the one embodiment of a graphical display of the primary BLAST search results for a particular nucleic acid sequence.

FIG. 8 shows one embodiment of a Web based graphical user interface.

FIG. 9 shows one embodiment of system output reported in spreadsheet format.

FIG. 10 shows one embodiment of system output reported in spreadsheet format.

FIG. 11 shows a schematic representation of one embodiment of the systems and programs of the present invention.

FIG. 12 shows a schematic representation of one embodiment of the systems ad programs of the present invention.

FIG. 13 shows one embodiment of a Web based graphical user interface.

FIG. 14 shows one embodiment of a Web based graphical user interface.

FIG. 15 shows one embodiment of a Web based graphical user interface.

FIG. 16 shows one embodiment of a Web based graphical user interface.

### DEFINITIONS

To facilitate an understanding of the present invention, a number of terms and phrases are defined below:

As used herein, the term “host cell” refers to any eukaryotic cell (e.g., mammalian, avian, amphibian, fish, insect, and plant), whether located in vitro or in vivo.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein, the term “genome” refers to the genetic material (e.g., chromosomes) of an organism or a host cell.

As used herein, “distinct biological samples” refers to two or more separate and discrete biological samples that are distinguished by either i) the sample type (e.g., same source, but different type), or ii) the source of the sample (e.g., same sample type, but different source). For example, in one embodiment, samples are taken from similar or identical biological tissues (e.g., a tissue sample from a first source and a second source, wherein said tissue is from the same organ) or similar organisms. In another embodiment, two or more sources are used, the first sample being derived from a first biological source (e.g., skin, liver, pancreas, prostate, blood cells, E. coli, etc.) that is suspected of being altered (e.g., diseased, or pathogenic), and the second sample (or in the case of multiple samples, a third, fourth, fifth, etc.) from a second source of the same type (e.g., liver cells compared to liver cells, E. coli compared E. coli., etc.). In one embodiment, the samples are derived from one or more discrete but similar biological tissues or organisms (e.g., a control(s)) that are believed to be normal (e.g., non-diseased, or non-pathogenic) when compared to the suspected altered biological samples derived from the first source. In another embodiment, different samples are taken from the same source (e.g., a first sample from one organ and a second sample from another organ, or a first sample from a disease free portion of an organ and a second sample from a diseased portion of the same organ). In preferred embodiments, a first biological sample is derived from a patient, or cell line, suspected of being cancerous (e.g., cancerous liver cells) are obtained, and a second, or more, biological sample derived from a patient, or cell line, that is not cancerous (e.g., non-cancerous liver cells) are likewise obtained.

As used herein, the term “native” (or wild type) when used in reference to a protein refers to proteins encoded by partially homologous nucleic acids so that the amino acid sequence of the proteins varies. As used herein, the term “variant” encompasses proteins encoded by homologous genes having both conservative and nonconservative amino acid substitutions that do not result in a change in protein function, as well as proteins encoded by homologous genes having amino acid substitutions that cause decreased (e.g., null mutations) protein function or increased protein function. While not intended to limit the invention, in a preferred embodiment, the sequences immobilized on the array used for hybridization (discussed above) are native sequences, or portions thereof.

As used herein, “transmembrane protein” refers to protein or polypeptide suspected of transverse the cell membrane and having at least a portion contacting the extracellular space.

The term “hydrophobic domain,” refers to portion or motif of a protein or polypeptide that cannot form favorable bonding (e.g., hydrogen bonds) with water molecules. A “hydrophobic domain” can be functionally defined as a domain that causes a portion of a peptide to be inserted into a membrane (e.g., in the lipid bilayer). Examples of such domains include coiled and helical polypeptide structures comprised of a high number of non-polar amino acids.

The term “gene” refers to a nucleic acid (e.g., DNA or RNA) sequence that comprises coding sequences necessary for the production of a polypeptide or precursor (e.g., proinsulin). The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and includes sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ untranslated sequences. The sequences that are located 3′ or downstream of the coding region and which are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage and polyadenylation.

As used herein, the term “structural gene” refers to a DNA sequence coding for RNA or a protein. In contrast, “regulatory genes” are structural genes that encode products that control the expression of other genes (e.g., transcription factors). The present invention contemplates that the sequences immobilized on the array used for hybridization (discussed above) can represent either structural or regulatory genes, or portions thereof.

“Nucleic acid sequence” and “nucleotide sequence” as used herein refer to an oligonucleotide or polynucleotide, and fragments or portions thereof, and to DNA or RNA of genomic or synthetic origin that may be single- or double-stranded, and represent the sense or antisense strand. As used herein, the terms “nucleic acid molecule encoding,” “DNA sequence encoding,” “DNA encoding,” “RNA sequence encoding,” and “RNA encoding” refer to the order or sequence of deoxyribonucleotides or ribonucleotides along a strand of deoxyribonucleic acid or ribonucleic acid. The order of these deoxyribonucleotides or ribonucleotides determines the order of amino acids along the polypeptide (protein) chain translated from the mRNA. The DNA or RNA sequence thus codes for the amino acid sequence.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

The term “nucleotide sequence of interest” refers to any nucleotide sequence (e.g., RNA or DNA), the identification or manipulation of which may be deemed desirable for any reason (e.g., treat disease, confer improved qualities, etc.), by one of ordinary skill in the art. Such nucleotide sequences include, but are not limited to, coding sequences, or portions thereof, of structural genes (e.g., reporter genes, selection marker genes, oncogenes, drug resistance genes, growth factors, etc.), and non-coding regulatory sequences that do not encode an mRNA or protein product (e.g., promoter sequence, polyadenylation sequence, termination sequence, enhancer sequence, etc.). In one embodiment, the nucleotide sequence of interest is one coding for a hydrophobic domain (or other membrane related motif).

As used herein, the term “protein of interest” refers to a protein encoded by a nucleic acid of interest. In one embodiment, the protein of interest is one comprising a hydrophobic domain (or other membrane related motif).

As used herein, the term “sample template” refers to nucleic acid originating from a sample that is analyzed for the presence of a target sequence of interest. In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

“Amplification” is defined as the production of additional copies of a nucleic acid sequence and is generally carried out using polymerase chain reaction technologies well known in the art (Dieffenbach C. W., and G. S., Dveksler, PCR Primer, a Laboratory Manual, Cold Spring Harbor Press, Plainview N.Y. [1995]). As used herein, the term “polymerase chain reaction” (“PCR”) refers to the method of K. B. Mullis U.S. Pat. Nos. 4,683,195 and 4,683,202, hereby incorporated by reference, that describes a method for increasing the concentration of a segment of a target sequence in a mixture of genomic DNA without cloning or purification. The length of the amplified segment of the desired target sequence is determined by the relative positions of two oligonucleotide primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”.

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

Amplification in PCR requires “PCR reagents” or “PCR materials”, that herein are defined as all reagents necessary to carry out amplification except the polymerase, primers and template. PCR reagents nominally include nucleic acid precursors (dCTP, dTTP etc.) and buffer.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is immobilized on solid support and capable of hybridizing to another sequence of interest (target). A probe may be single-stranded or double-stranded. Probes are useful in the detection, identification and isolation of particular gene sequences.

As used herein, the term “target” refers to sequence of nucleotides, where occurring naturally as in a purified RNA, purified restriction digest, or produced synthetically or by PCR amplification that is capable of hybridizing to the probe sequences. A target may be single-stranded or double-stranded. It is contemplated that any target used in the present invention will be labeled with any “reporter molecule,” so that it is detectable using any detection system, including, but not limited to enzyme (e.g., ELISA, as well as enzyme-based histochemical assays), fluorescent, radioactive, and luminescent systems. The present invention is not limited to any particular detection system or label.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct the transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in that only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The terms “homology” and “percent identity” when used in relation to nucleic acids refers to a degree of complementarity. There may be partial homology (i.e., partial identity) or complete homology (i.e., complete identity). A partially complementary sequence is one that at least partially inhibits a completely complementary sequence from hybridizing to a target nucleic acid sequence and is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe (i.e., an oligonucleotide) that is capable of hybridizing to another oligonucleotide of interest) will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that lacks even a partial degree of complementarity (e.g., less than about 30% identity); in the absence of non-specific binding the probe will not hybridize to the second non-complementary target.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tm of the formed hybrid, and the G:C ratio within the nucleic acids. A single molecule that contains pairing of complementary nucleic acids within its structure is said to be “self-hybridized.”

As used herein the term “hybridization complex” refers to a complex formed between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., Cot or Rot analysis) or between one nucleic acid sequence present in the solution and another nucleic acid sequence immobilized to a solid support (e.g., a nylon membrane or a nitrocellulose filter as employed in Southern and Northern blotting, dot blotting, or a glass slide as employed in in situ hybridization, including FISH [fluorescent in situ hybridization], arrays and microarrays, and the like).

As used herein the term “stringency” is used in reference to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required with nucleic acids that are derived from organisms that are genetically diverse, as the frequency of complementary sequences is usually less. While the present invention can be practiced utilizing low stringency conditions, in a preferred embodiment, medium to high stringency conditions are employed.

“High stringency conditions” when used in reference to nucleic acid hybridization comprise conditions equivalent to binding or hybridization at 42° C. (e.g., overnight) in a solution consisting of 5×SSPE (43.8 g/l NaCl, 6.9 g/l NaH2PO4H2O and 1.85 g/l EDTA, pH adjusted to 7.4 with NaOH), 0.5% SDS, 5× Denhardt's reagent [50× Denhardt's contains per 500 ml, 5 g Ficoll (Type 400, Pharamcia), 5 g BSA (Fraction V; Sigma)], 100 mg/ml denatured salmon sperm DNA and 50% (V/V) of formamide. The hybridized DNA samples are then washed twice in a solution comprising 2×SSPE, 0.1% SDS at room temperature followed by 0.1×SSPE, 1.0% SDS at 42° C. when a probe of about 500 nucleotides in length is employed. The art knows conditions that promote hybridization under conditions of high stringency (e.g., increasing the temperature of the hybridization and/or wash steps, the use of formamide in the hybridization solution, etc.).

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH2PO4H2O and 1.85 g/l EDTA, pH adjusted to 7.4 with NaOH), 0.5% SDS, 5× Denhardt's reagent [50× Denhardt's contains per 500 ml, 5 g Ficoll (Type 400, Pharamcia), 5 g BSA (Fraction V; Sigma)] 100 mg/ml denatured salmon sperm DNA, and 50% (V/V) formamide, followed by washing in a solution comprising 1.0×SSPE, 1.0% SDS at room temperature followed by 2×SSPE, 0.1% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH2PO4H2O and 1.85 g/l EDTA, pH adjusted to 7.4 with NaOH), 0.1% SDS, 5× Denhardt's reagent [50× Denhardt's contains per 500 ml, 5 g Ficoll (Type 400, Pharamcia), 5 g BSA (Fraction V; Sigma)], 100 g/ml denatured salmon sperm DNA, and 50%(V/V) formamide followed by washing in a solution comprising 5×SSPE, 0.1% SDS at 42° C. when a probe of about 500 nucleotides in length is employed. In addition, the art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions.

When used in reference to a single-stranded nucleic acid sequence, the term “substantially homologous” refers to any probe that can hybridize (i.e., it is the complement of) the single-stranded nucleic acid sequence under conditions of low stringency as described above. A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other. The present invention is not limited to the situation where hybridization takes place only between completely homologous sequences. In some embodiments, hybridization takes place with substantially homologous sequences.

As used herein, the term “Tm” is used in reference to the “melting temperature” of a nucleic acid. The melting temperature is the temperature at which a population of double-stranded nucleic acid molecules becomes half dissociated into single strands. The equation for calculating the Tm of nucleic acids is well known in the art. As indicated by standard references, a simple estimate of the Tm value may be calculated by the equation: Tm=81.5+0.41(% G+C), when a nucleic acid is in aqueous solution at 1 M NaCl. (See e.g., Anderson and Young, Quantitative Filter Hybridization, in Nucleic Acid Hybridization [1985]). Other references include more sophisticated computations that take structural as well as sequence characteristics into account for the calculation of Tm. While the present invention can be practiced using hybridization temperatures below the calculated Tm, in a preferred embodiment, the hybridization temperature employed is at or above the calculated Tm.

The term “Southern blot” refers to the analysis of DNA on agarose or acrylamide gels to fractionate the DNA according to size, followed by transfer and immobilization of the DNA from the gel to a solid support, such as nitrocellulose or a nylon membrane. The immobilized DNA is then probed with a labeled oligodeoxyribonucleotide probe or DNA probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A laboratory Manual, Cold Spring Harbor Press, NY, pp. 9.31-9.58 [1989]).

The term “Northern blot” as used herein refers to the analysis of RNA or mRNA by electrophoresis of RNA or mRNA on agarose gels to fractionate the RNA or mRNA according to size followed by transfer of the RNA or mRNA from the gel to a solid support, such as nitrocellulose or a nylon membrane. The immobilized RNA or mRNA is then probed with a labeled oligodeoxyribonucleotide probe or DNA probe to detect RNA or mRNA species complementary to the probe used. Northern blots are a standard tool of molecular biologists (J. Sambrook, J. et al., supra, pp. 7.39-7.52 [1989]).

The term “reverse Northern blot” as used herein refers to the analysis of DNA by electrophoresis of DNA on agarose gels to fractionate the DNA on the basis of size followed by transfer of the fractionated DNA from the gel to a solid support, such as nitrocellulose or a nylon membrane. The immobilized DNA is then probed with a labeled oligo-ribonucleotide probe or RNA probe to detect DNA species complementary to the ribo probe used.

As used herein the term, the term “in vitro” refers to an artificial environment and to processes or reactions that occur within an artificial environment. In vitro environments can consist of, but are not limited to, test tubes and cell cultures. The term “in vivo” refers to the natural environment (e.g., an animal or a cell) and to processes or reaction that occur within a natural environment.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acids are nucleic acids present in a form or setting that is different from that in which they are found in nature. In contrast, non-isolated nucleic acids are nucleic acids such as DNA and RNA that are found in the state in which they exist in nature.

As used herein, the term “purified” or “to purify” refers to the removal of undesired components from a sample. As used herein, the term “substantially purified” refers to molecules, either nucleic or amino acid sequences, that are removed from their natural environment, isolated or separated, and are at least 60% free, preferably 75% free, and most preferably 90% free from other components with which they are naturally associated. An “isolated polynucleotide” is therefore a substantially purified polynucleotide.

The terms “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. Also included within this term are prokaryotic organisms that are gram negative or gram positive. “Gram negative” and “gram positive” refer to staining patterns with the Gram-staining process that is well known in the art. (See e.g., Finegold and Martin, Diagnostic Microbiology, 6th Ed., CV Mosby St. Louis, pp 13-15 [1982]). “Gram positive bacteria” are bacteria that retain the primary dye used in the Gram stain, causing the stained cells to appear dark blue to purple under the microscope. “Gram negative bacteria” do not retain the primary dye used in the Gram stain, but are stained by the counterstain. Thus, gram negative bacteria appear red.

As used herein, the term “protein encoded by an oncogene” refers to proteins that cause, either directly or indirectly, the neoplastic transformation of a host cell. Examples of oncogenes include, but are not limited to, the following genes: src, fps, fes, fgr, ros, H-ras, abl, ski, erbA, erbB, fms, fos, mos, sis, myc, myb, rel, kit, raf, K-ras, and etc.

As used herein, the term “antigen binding protein” refers to proteins that bind to a specific antigen. “Antigen binding proteins” include, but are not limited to, immunoglobulins, including polyclonal, monoclonal, chimeric, single chain, and humanized antibodies, Fab fragments, F(ab′)₂ fragments, and Fab expression libraries. Various procedures known in the art are used for the production of polyclonal antibodies. For the production of antibody, various host animals can be immunized by injection with the peptide corresponding to the desired epitope including but not limited to rabbits, mice, rats, sheep, goats, etc. In a preferred embodiment, the peptide is conjugated to an immunogenic carrier (e.g., diphtheria toxoid, bovine serum albumin (BSA), or keyhole limpet hemocyanin (KLH)). Various adjuvants are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

For preparation of monoclonal antibodies, any technique that provides for the production of antibody molecules by continuous cell lines in culture may be used (See e.g., Harlow and Lane, Antibodies: A Laboratory Manual, Cold Spring Harbor Laboratory Press, Cold Spring Harbor, N.Y.). These include, but are not limited to, the hybridoma technique originally developed by Köhler and Milstein (Köhler and Milstein, Nature, 256:495-497 [1975]), as well as the trioma technique, the human B-cell hybridoma technique (See e.g., Kozbor et al., hnmunol. Today, 4:72 [1983]), and the EBV-hybridoma technique to produce human monoclonal antibodies (Cole et al., in Monoclonal Antibodies and Cancer Therapy, Alan R. Liss, Inc., pp. 77-96 [1985]).

According to the invention, techniques described for the production of single chain antibodies (U.S. Pat. No. 4,946,778; herein incorporated by reference) can be adapted to produce specific single chain antibodies as desired. An additional embodiment of the invention utilizes the techniques known in the art for the construction of Fab expression libraries (Huse et al., Science, 246:1275-1281 [1989]) to allow rapid and easy identification of monoclonal Fab fragments with the desired specificity.

Antibody fragments that contain the idiotype (antigen binding region) of the antibody molecule can be generated by known techniques. For example, such fragments include but are not limited to: the F(ab′)₂ fragment that can be produced by pepsin digestion of an antibody molecule; the Fab′ fragments that can be generated by reducing the disulfide bridges of an F(ab′)₂ fragment, and the Fab fragments that can be generated by treating an antibody molecule with papain and a reducing agent.

Genes encoding antigen-binding proteins can be isolated by methods known in the art. In the production of antibodies, screening for the desired antibody can be accomplished by techniques known in the art (e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western Blots, precipitation reactions, agglutination assays (e.g., gel agglutination assays, hemagglutination assays, etc.), complement fixation assays, immunofluorescence assays, protein A assays, and immunoelectrophoresis assays, etc.) etc.

As used herein, “microarray” refers to a substrate with a plurality of molecules (e.g., nucleotides) bound to its surface. Microarrays, for example, are described generally in Schena, “Microarray Biochip Technology,” Eaton Publishing, Natick, Mass., 2000. Additionally, the term “patterned microarrays” refers to microarray substrates with a plurality of molecules non-randomly bound to its surface (e.g., immobilized nucleic acid sequences in a grid or x/y arrangement).

As used herein, the term “detector” and “detector system” refer to a device that generates an output signal when irradiated with energy (e.g., optical, radiation, and the like). Thus, in its broadest sense the term detector system is taken to mean a device for converting energy from one form to another for the purpose of measurement of a physical quantity or for information transfer. In a preferred embodiment, the detector is configured to receive information (e.g., fluorescence) from a microarray.

As used herein the term “program”, when used as a noun, refers to an organized list of instructions that when executed (i.e., performed by the computer) causes a computer (or other processor) to behave in a predetermined manner (e.g., to design a set of oligonucleotides that hybridize to one to one or more nucleic acids of interest). Programs consist of modules, each of which contains one or more routines/subroutines (i.e., a section of a program that performs a particular task). As used herein, the term “routine” is synonymous with the terms procedure, function, and subroutine. In preferred embodiments, the programs described herein are written in a high-level programming language, for example, C, C++, Pascal, BASIC, FORTRAN, COBOL, or LISP, and the like. In preferred embodiments, the present invention provides a command-driven or menu-driven program and method for designing and generating oligonucleotides that hybridize to user entered nucleic acid sequences.

As used herein, the terms “processor,” “computer processor,” “computer,” and “central processing unit” or “CPU” are used interchangeably to refer to a device that is able to read a program from a computer memory (e.g., ROM or other computer memory) and perform a set of functions according to the program.

As used herein, the terms “memory device”, “computer memory device”, or “computer memory” refer to any storage media readable by a computer processor. Examples of computer memory include, but are not limited to, RAM, ROM, computer chips, digital video disc (DVDs), compact discs (CDs), hard disk drives (HDDs), magnetic tape, and the like.

As used herein, the term “computer readable medium” refers to any device or system for storing and providing information (e.g., data and instructions) to a computer processor. Examples of computer readable media include, but are not limited to, DVDs, CDs, HDDs, magnetic tape and servers for streaming media over networks.

As used herein, the term “information” refers to any collection of facts (e.g., nucleic acid or amino acid sequences) and data and any combination thereof. In reference to information stored by, or processed using, computer systems, the term refers to any data stored in a format (e.g., analog, digital, optical, etc.) readable by a computer processor. In preferred embodiments, information refers to nucleic acid sequences.

The term “operably linked,” in one sense, when used in reference to the operation of the disclosed systems and programs for generating oligonucleotides refers to the execution (e.g., performance by a computer) of a computer program by a computer processor to produce a desired result (e.g., the generation of oligonucleotide probes that hybridize to target nucleotides in a biological sample). In another sense, the term “operably linked,” when used in reference to the operation of the disclosed systems and programs for generating oligonucleotides, refers to computer hardware devices and other apparatuses (e.g., computer memory devices, computer monitors, printers, keyboards, computer mouse, communications links [e.g., fiber optic cables, telephone lines, infrared beams [IR], LANs, satellite links, the Internet, and the like], oligonucleotide synthesizing machines, microarray readers [e.g., CCDs], etc.) configured to receive and/or exchange information with the disclosed systems and programs for generating oligonucleotides. In preferred embodiments, operably linked computer hardware devices and other apparatuses are configured to receive and/or exchange information with a computer program stored in computer readable memory associated with a computer processor via, for example, wires or cables, computer cards and boards, circuits, communication links [e.g., fiber optic cables, telephone lines, IR, LANs, satellite links, the Internet], etc., and any necessary devices or subroutines stored in computer readable memory. In preferred embodiments, the oligonucleotide generating program of the present invention is operably linked to one or more information output and input devices (e.g., printers, computer monitors, keyboards, computer mice, voice recognition devices (microphones), computer memory devices, oligonucleotide synthesizing machines, communications connections, and a computer processor (e.g., in preferred embodiments the program is stored in computer memory).

As used herein the terms “user,” and “system user” when used in reference to controlling the operation of a computer program (e.g., a software program stored in computer memory that generates oligonucleotide sequences), refers to a person, or a second computer program or system (e.g., a software program stored in computer memory) that controls the operation of the first computer program by selecting and/or entering system operation instructions and information.

As used herein the term “user interface” refers to the bi-directional junction between a user and a computer program configured for the exchange (e.g., input and/or output) of information and operating instructions. The “user interface” allows the user to input commands (e.g., instructions) that direct a computer program, hardware device, or other apparatus to perform specific tasks. As used herein the term “graphical user interface,” or (GUI), refers to a user interface that takes advantage of the computer's graphics generating capabilities. “User interfaces,” which optionally often include computer operating systems such as Microsoft Windows and Apple Macintosh OSx, include, but are not limited to, the following components for conveying user input to a program: pointing devices (e.g., mouse, trackball, digitized pens, etc.); icons; a virtual desk top; menus and the like.

As used herein, the term “dialogue entry box(es)” when used in reference to the oligonucleotide generating systems and programs disclosed herein, is used consistently with accepted usage and definition in the art. For example, in one embodiment, the system user inputs information (e.g., user set parameters or nucleic acid sequences) and data into the disclosed systems and programs via one or more dialogue entry boxes.

The term “user set parameters,” as used herein, refers to the information entered into the program and systems by the user (e.g., through a GUI and/or one or more dialogue entry boxes) that control the variable routines and subroutines that comprise the systems and programs disclosed herein for generating oligonucleotide sequences. For example, the system user can enter information instructing the program to consider, for example: the length of the oligonucleotides generated; the number of oligonucleotides generated per nucleic acid sequence entered into the system; the minimum (A+T)/(G+C) ratio of the oligonucleotides generated; 4) the minimum proximity among multiple oligonucleotides generated; 5) the system user may elect to have the system remove oligonucleotides generated that contain simple repeats (e.g., homoploymers, di-, or tri-nucleotide repeats, and direct or inverted repeats); and the amount of time allowed for conducting BLAST searches to compare the oligonucleotides generated to one or more genomic databases.

As used herein, the term “database” refers to one or more collections of information arranged for ease of retrieval by, for example, being stored temporarily (or permanently) in a computer readable memory. In preferred embodiments of the present invention, a database comprises nucleic acid sequence information (e.g., ribonucleic acid or deoxyribonucleic acid sequences, including any modified bases or bases analogs contained therein).

As used herein, the term “total sequence information set” refers to a grouping of information (e.g., one or more nucleic acid sequences and any other related information) stored in a computer readable memory device (or other computer readable medium), that comprises, but is not limited to, all of the nucleic acid sequences entered by the system user (e.g., using a GUI) into the present system and programs that the user wants to analyze during a particular session. In preferred embodiments, the “total sequence information set” comprises one or more nucleic acid sequences that encode transmembrane proteins or polypeptides. In particularly preferred embodiments, the “total sequence information set” is configured to communicate with the program and computer processor comprising the present invention.

As used herein, the term “single nucleotide sequence information set” refers to a particular nucleic acid sequence and related information (e.g., a sequence identifier, for example, a FASTA nucleic acid sequence code) selected (i.e., sequentially, randomly, or rationally) by the systems and programs disclosed herein from the total sequence information set for further manipulation. In preferred embodiments, the “single nucleotide sequence information set,” comprises one nucleic acid sequence (and any related information) at a time sequentially selected from the total information set sequence database. In particularly preferred embodiments, the “single nucleotide sequence information set” is configured to communicate with the program and computer processor comprising the present invention (e.g., total sequence information set and the first filter).

As used herein, the term “first filter” refers to a subroutine of the computer program for generating oligonucleotides disclosed herein that is configured to manipulate nucleic acid sequences from the total sequence information set and/or the single nucleotide sequence information set such that the sequence complexity and/or the presence of secondary structure in a selected nucleic acid sequence is determined. In preferred embodiments, the “first filter” sequentially analyzes nucleic acid sequences comprising the single nucleotide sequence information set. In other embodiments, the “first filter” sequentially selects nucleic acid sequences from the total sequence information set directly. In particularly preferred embodiments, the “first filter” is configured to communicate with the program and computer processor comprising the present invention (e.g., the single nucleotide sequence information set, the total sequence information set, and the total oligonucleotides of all sequences information set).

As used herein, the term “total oligonucleotides of all sequences information set” refers to the set of information (e.g., the one or more nucleic acid sequences analyzed for sequence complexity and/or the presence of secondary structure) generated by operation of the first filter of the present invention along with any other information related to these nucleic acid sequences. In preferred embodiments, the “total oligonucleotides of all sequences information set” comprises the aggregate of information generated by the manipulations of the first filter described herein. In particularly preferred embodiments, the “total oligonucleotides of all sequences information set” is configured to communicate with the program and computer processor comprising the present invention (e.g., the first filter and the primary oligonucleotides per sequence information set).

As used herein, the term “primary oligonucleotides per sequence information set” refers to the information generated by the first filter contained within the total oligonucleotides of all sequences information set that corresponds to a particular nucleic acid sequence of interest entered by the system user. Thus, in preferred embodiments, the total oligonucleotides of all sequences information sets comprise one or more primary information sets of oligonucleotides per sequence entered by the system user. In particularly preferred embodiments, the “primary oligonucleotides per sequence information set” is configured to communicate with the program and computer processor comprising the present invention (e.g., total oligonucleotides of all sequences information set and the second filter).

As used herein, the term “second filter” refers to a subroutine of the computer program for generating oligonucleotides disclosed herein that is configured to analyze the individual oligonucleotides comprising the primary oligonucleotides per sequence information set to determine: 1) uniqueness in genome database (e.g., conduct a BLAST search of the candidate oligonucleotide against a genomic database); 2) rank based on proximity to the 3′ end of the single nucleotide sequence entered; and 3) rank based on (A+T)/(G+C) ratio. In particularly preferred embodiments, the “second filter” is configured to communicate with the program and computer processor comprising the present invention (e.g., primary oligonucleotides per sequence information set and the secondary set of oligonucleotides per sequence information set).

The term “secondary set of oligonucleotides per sequence information set,” as used herein, refers to the set of information (e.g., the one or more nucleic acid sequences analyzed by the second filter and any related information) analyzed by the second filter and ranked by the system as to provide the best candidate oligonucleotides for hybridization to a particular nucleic acid sequence of interest entered into the system. In particularly preferred embodiments, the secondary set of oligonucleotides per sequence information set is configured to communicate with the program and computer processor comprising the present invention (e.g., the second filter).

As used herein, the term “list of sequences with no oligonucleotides generated,” when used in reference the disclosed oligonucleotide generating systems and programs, refers to a computer memory configured to store nucleic acid sequences, and/or sequence identifiers (e.g., GenBank accession numbers), rejected by the program because the sequences failed to generate desired system output (e.g., one or more unique oligonucleotides that meet the user set parameters).

The term “processing user information”, when used in reference to the operation of the disclosed systems and programs, refers to the execution of said program as directed by the information and operational parameters entered by the system user (e.g., the nucleic acid sequences entered to be analyzed along with, but not limited to, such information as: 1) desired length of the oligonucleotides generated; 2) the number of oligonucleotide generated per nucleic acid sequence entered into the system; 3) the minimum (A+T)/(G+C) ratio of the oligonucleotides generated; 4) the minimum proximity among multiple oligonucleotides generated; and 5) the system user may elect to have the system remove oligonucleotides generated that contain simple repeats (e.g., homoploymers, di-, or tri-nucleotide repeats, and direct or inverted repeats.

As used herein, the term “oligonucleotide candidates,” or “oligonucleotide probe candidates,” refer to short oligonucleotide sequences of about 25 . . . 50 . . . 75 . . . 100, or more nucleotides in length, and in preferred embodiments, about 50 nucleotides in length, that are manipulated (e.g., analyzed by the first and second screens) by the programs and systems of the present invention. Accordingly, as used herein, the terms “output”, or “system output” refer to the oligonucleotide sequences (and any additional information) generated by the systems and programs (e.g., system results).

The term “Internet”, as used herein, refers to a collection of interconnected (public and/or private) networks that are linked together by a set of standard protocols (such as TCP/IP and HTTP) to form a global, distributed information network. While this term is intended to refer to what is now commonly known as the Internet, it is also intended to encompass variations that may be made in the future, including changes and additions to existing standard protocols. In certain embodiments, the systems and programs of the present invention for designing oligonucleotides are accessible via the Internet (i.e., World Wide Web) over a communication network.

As used herein, the terms “World Wide Web” or “Web” refer generally to both (i) a distributed collection of interlinked, user-viewable hypertext documents (commonly referred to as Web documents or Web pages) that are accessible via the Internet, and (ii) the client and server software components that provide user access to such documents using standardized Internet protocols. Currently, the primary standard protocol for allowing applications to locate and acquire Web documents is HTTP, and the Web pages are encoded using HTML. However, the terms “Web” and “World Wide Web” are intended to encompass future markup languages and transport protocols that may be used in place of (or in addition to) HTML and HTTP.

As used herein, the term “Web Site” refers to a computer system that serves informational content over a network using the standard protocols of the World Wide Web. Typically, a Web site corresponds to a particular Internet domain name and includes the content associated with a particular organization or application (e.g., a system for generating oligonucleotide probe sets). As used herein, the term is generally intended to encompass both (i) the hardware/software server components that serve the informational content over the network, and (ii) the “back end” hardware/software components, including any non-standard or specialized components, that interact with the server components to perform services for Web site users. In certain embodiments, the systems and programs of the present invention are posted on one or more Web sites and are thus potentially accessible by system users around the world.

As used herein, the term “HTML” refers to HyperText Markup Language that is a standard coding convention and set of codes for attaching presentation and linking attributes to informational content within documents. During a document authoring stage, the HTML codes (referred to as “tags”) are embedded within the informational content of the document. When the Web document (or HTML document) is subsequently transferred from a Web server to a browser, the codes are interpreted by the browser and used to parse and display the document. In specifying how the Web browser is to display the document, HTML tags can be used to create links to other Web documents (commonly referred to as “hyperlinks”). In preferred embodiments, the systems and programs of the present invention provide system output (i.e., oligonucleotide sequences generated by the system, and any other additional information) to the system user via one or more Web pages created in the standard HTNL format. As used herein, the term “HTTP” refers to HyperText Transport Protocol that is the standard World Wide Web client-server protocol used for the exchange of information (such as HTML documents, and client requests for such documents) between a browser and a Web server. HTTP includes a number of different types of messages that can be sent from the client to the server to request different types of server actions. For example, a “GET” message, which has the format GET, causes the server to return the document or file located at the specified URL.

As used herein, the term “URL” refers to Uniform Resource Locator that is a unique address that fully specifies the location of a file or other resource on the Internet. The general format of a URL is protocol://machine address:port/path/filename. The port specification is optional, and if the user enters none, the browser defaults to the standard port for whatever service is specified as the protocol. For example, if HTTP is specified as the protocol, the browser will use the HTTP default port of 80. In certain embodiments, the present invention contemplates that one or more URLs can be devised to identify and distinguish the systems and programs of the present invention when they are made available on the Internet.

As used herein, the term “cookies” refers to a technology that enables a Web server to retrieve information from a user's computer that reveals prior browsing activities of the user. The informational item stored on the user's computer (typically on the hard drive) is commonly referred to as a “cookie.” Many standard Web browsers support the use of cookies. In certain embodiments of the present invention, the systems and programs disclosed herein are configured to send one or more cookies to users accessing the program from a remote location (e.g., via a communication network).

As used herein, the term “communication network” refers to any network that allows information to be transmitted from one location to another. For example, a communication network for the transfer of information from one computer to another includes any public or private network that transfers information using electrical, optical, satellite transmission, and the like. Two or more devices that are part of a communication network such that they can directly or indirectly transmit information from one to the other are considered to be “in electronic communication” with one another. A computer network containing multiple computers may have a central computer (“central node”) that processes information to one or more sub-computers that carry out specific tasks (“sub-nodes”). Some networks comprise computers that are in “different geographic locations” from one another, meaning that the computers are located in different physical locations. Some embodiments of the present invention contemplate using communications networks to make input to, or output from the present systems and programs more rapid and convenient for system users. For example, certain embodiments contemplate, as described above, making one or more system Web pages available over the Internet to system users.

General Description of the Invention

Generally the drug development process is slow and costly. For example, it may take up to 15 years and cost nearly $500 million to bring one therapeutic compound to market. Most of the resources and efforts of drug discovery programs are spent studying the biological activities of disease targets and identifying lead compounds. A better and more meaningful compound screening protocol would be designed based on an understanding of the biological functions of the therapeutic target. However, it is difficult to completely understand the function of a protein and design a comprehensive screening protocol. Therefore, chemical based drug development usually focuses on small numbers of well-studied gene families, such as Proteases, Kinases, and G-protein coupled receptors.

Recent advances in antibody production, stability, humanization, and delivery methods suggest that humanized antibodies can effectively be used as therapeutic agents. One advantage of antibody therapeutics over chemical compounds is their specificity toward target proteins. The high specificity of antibodies against target proteins implies that antibody based therapeutics can be designed that are less toxic and have fewer side effects than chemical based drugs. Moreover, due to the lack of laborious compound screening processes, the time required to produce antibody-based therapeutics is much shorter than for chemical based drugs. However, effective antibody therapeutics must be able to access their intended targets (e.g., proteins). This means that cell membrane proteins are preferential targets for antibody therapeutics.

In preferred embodiments, the present invention provides systems and methods for determining the differential expression of proteins, and in particular, for determining the differential expression of cell membrane proteins to identify potential drug targets (e.g., antibody therapeutics). The present invention also provides systems and methods for determining the differential expression of genes encoding cell membrane/wall proteins of microorganisms (e.g., a bacterium) for determining microorganism serotypes and potential pathogenicity.

Normal cells use various membrane proteins receptors to respond to external stimuli, to regulate their growth and division, and to interact and communicate with each other. Cancer cells, on the other hand, have lost the ability to regulate growth and cell division. Thus, it is reasonable to assume that the composition of the cell membrane proteins on cancer cells are different from their normal cellular counterparts to reflect the miscommunication and altered growth characteristics of cancer cells. Despite gaps in the understanding of how and why cancer cells arise and spread to distal organs, the therapeutic goal of cancer treatment is simple: to eliminate the cancerous cells from the patient's body.

Existing approaches for producing antibodies to cell membrane proteins require isolating two pools of RNAs, one pool from abnormal cells (e.g., cancer cells) and a second pool from normal cells, differentially labeling the two pools (e.g., by distinguishable fluorescent markers), combining the labeled pools, and performing one or more experiments to identify differentially-expressed genes. However, the process of identifying and selecting cell membrane proteins from experimental results (e.g., after the assay is complete) is laborious and unreliable.

In contrast, the methods described herein for identifying potentially differentially expressed cell membrane proteins differs from existing approaches. The present invention contemplates that differentially expressed membrane-bound cellular proteins on diseased cells (e.g., cancer cells) provide targets for possible therapeutic intervention (e.g., antibody therapeutics).

The present invention provides systems and methods for generating therapeutic antibodies to one or more membrane proteins identified as likely to correspond to differentially expressed genes. In preferred embodiments, antibodies are screened to evaluate their ability to bind to differentially expressed membrane proteins. For example, in some embodiments, the present invention provides methods employing various computation tools to pre-select genes encoding cell membrane proteins that can be recognized by antibodies. In certain of these embodiments, one or more genomic libraries are prescreened to select only those genes predicted to encode cell membrane proteins. The present invention provides various computational methods that allow the user to minimize: 1) the occurrence of competitive hybridization between distinct probe sequences; 2) nonspecific binding between “targets” and “probes;” and 3) formation of secondary structures in probe sequences, thus reducing the time and expense required to identify potentially differentially-expressed cell membrane proteins. The preselection step is not intended to be limited however to the computation tools described above.

The present invention further provides systems and methods for designing oligonucleotide-binding sequences that hybridize to preselected nucleotide sequences. In preferred embodiments, these systems and methods are used to generate sets of nucleic acid sequences that are then arrayed to provide hybridization probes for RNA isolated from one or more distinct biological samples. In preferred embodiments of the present invention, oligonucleotide sequences are arrayed and screened for expression level differences between various developmental or disease stages.

In contrast to the existing approaches for producing cDNA fragments for microarrays (e.g., cDNA library screening and PCR), the present invention provides methods and systems for efficiently designing oligonucleotide sequence arrays that represent a sequence of interest such as sequences encoding particular domains (e.g., hydrophobic domains). The oligonucleotide sequences provided by the systems and methods of the present invention comprise from about 10 to 200 bases. In preferred embodiments, the oligonucleotide sequences comprise from about 20 to 100 bases. In particularly preferred embodiments, the oligonucleotide sequences comprise between about 25 and 50 bases.

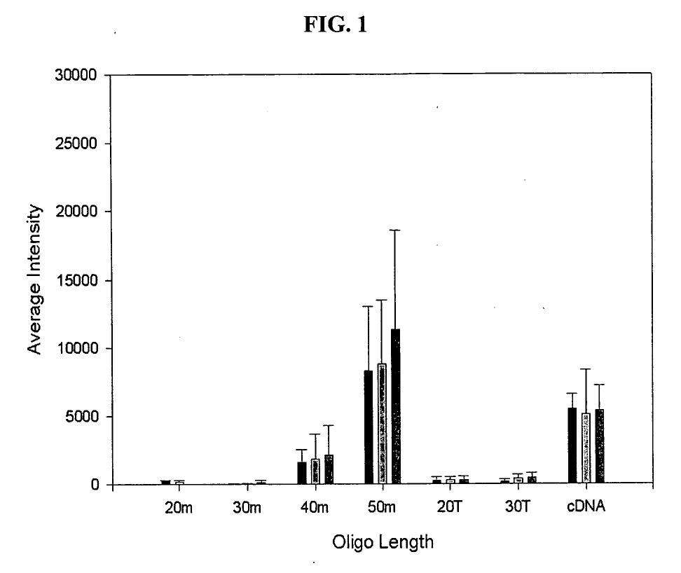

FIG. 1 shows a comparison of the hybridization sensitivity of the oligonucleotide sequences designed by the systems and methods of the present invention compared to corresponding cDNA fragments isolated from a cDNA library. Briefly, the lengths of the oligonucleotides sequences are provided on the bottom of the figure. The captions: 20 m, 30 m, 40 m, and 50 m represent 20-, 30-, 40-, and 50-nucleotide long oligonucleotide sequences, respectively. The captions: 20T and 30T represent the 20 m oligonucleotide sequence comprising additional 20- or 30-nucleotide long polyT sequences at their 5′ ends to anchor the oligonucleotides.

The present systems and methods for designing oligonucleotide sequences are not intended to be limited however to preselecting nucleotide sequences that encode cell membrane proteins. Indeed, the present invention provides systems and methods for designing oligonucleotide sequences to hybridize to any group of preselected nucleic acid sequences. The present invention further provides methods and systems for designing oligonucleotides sequences that hybridize to a variety of cellular genes, including, but not limited to, gene encoding signaling proteins, structural proteins, regulatory proteins, enzymes (e.g., kinases and proteases) and the like. In preferred embodiments, these oligonucleotide sequences are arrayed on solid surfaces (e.g., microchips, beads, culture plates, microtiter plates, glass slides, metal plates, membranes, sol-gels, etc.). The present invention contemplates detecting the differential expression of genes in any two biological samples or organisms.

Detailed Description of the Invention

The present invention relates to the identification of expressed genes, and in particular, to systems and methods for differentiating between the expression of genes encoding cell membrane proteins in two or more biological samples using differential gene expression analysis methods. In one embodiment, the present invention contemplates that the differential expression of cell membrane proteins indicates whether the sample (e.g., cells, tissues, bodily fluids) is diseased or pathogenic.

The present invention also provides methods and systems for designing oligonucleotide sequences that share one or more common features (e.g., code for a hydrophobic domain, although not necessarily the same hydrophobic domain). In one preferred embodiment, the oligonucleotide sequences are subsequently attached to an array.

The various embodiments of the present invention are described in more detail in the following sections: I) Overview of Techniques for Differential Gene Expression Analysis; II) Computational Approaches To Preselecting Nucleic Acid Probe Sequences; III) Approaches To Microarrays; IV. Approaches to Generating Oligonucleotide Primer Pairs; V) Approaches to Preparing Samples For Gene Expression Analysis; VI) Approaches To Antibody Generation and Humanization; VII) Verification Of Antibody Binding Affinity In Animal Models; and VIII) Pharmaceutical Compositions.

I. Overview of Techniques for Differential Gene Expression Analysis

Identifying differences in gene expression between biological samples is not trivial. One approach involves the production of a so-called “subtractive cDNA library.” A subtractive cDNA library contains cDNA clones corresponding to mRNAs present in one sample and not present in another (e.g., present in a particular species, tissue or cell and not present in another species, tissue or cell). See generally, Current Protocols in Molecular Biology, Section 5.8.9 (1990). In the protocol, cDNA containing the gene(s) of interest [“+cDNA”] is prepared with EcoRI ends and the cDNA not containing the gene(s) of interest [“−cDNA”] is prepared with blunt ends. The +cDNA is mixed with a 50-fold excess of −cDNA inserts and the mixture is heated to make the DNA single-stranded. Thereafter, the mixture is cooled to allow for hybridization. Annealed cDNA inserts are ligated to a vector and transfected. In theory, the only +cDNA likely to be double-stranded with an EcoRI site at each end are those not hybridized to something in the −cDNA preparation; in other words, where a complementary sequence is in the −cDNA preparation, the sequence will not be transfected. Thus, only sequences to the +cDNA preparation will be cloned and amplified.

The subtraction approach is tedious. Moreover the hybridizations and library production with a small amount of RNA are technically artful.

The second approach to identifying differentially expressed genes involves the use of arbitrary primer sets to on mRNA or during polymerase chain reaction (DDRT-PCR). The polymerase chain reaction is described by Mullis, et al., in U.S. Pat. Nos. 4,683,195, 4,683,202 and 4,965,188, hereby incorporated by reference. Briefly, the PCR process consists of introducing a molar excess of two oligonucleotide primers to the DNA mixture containing the desired target sequence. The two primers are complementary to their respective strands of the double-stranded sequence. The mixture is denatured and then allowed to hybridize. Following hybridization, the primers are extended with a thermostable DNA polymerase so as to form complementary strands. The steps of denaturation, hybridization, and polymerase extension can be repeated as often as needed to obtain a relatively high concentration of a segment of the desired target sequence.

In the case of DDRT-PCR, the target is mRNA; the mRNA is, however, treated with reverse transcriptase in the presence of oligo(dT) primers to make cDNA prior to the PCR process. The PCR is carried out at low stringency with short random primers in combination with the oligo(dT) primer used for cDNA synthesis. In theory, since only mRNA is (indirectly) amplified, only the expressed genes are amplified. Where two samples are to be compared, the amplified products are placed in side-by-side lanes of a gel; following electrophoresis, the PCR products can be compared or “differentially displayed.”

DDRT-PCR, while an improvement over subtractive hybridization, has a number of drawbacks. The use of arbitrary primers can cause faint banding at essentially every position of the gel. The process is generally biased toward high-copy number genes.

II. Computational Approaches to Preselecting Nucleic Acid Probe Sequences

In one embodiment, the present invention provides systems and methods for selecting (i.e., preselection step) oligonucleotides (genes) that potentially encode membrane proteins from sequence databases by using one or more transmembrane domain predicting programs. A number of databases, such as, the ensemble predicted unconfirmed protein database (http://www.ensembl.org/); the GenBank non-redundant (nr) protein database (http://www.ncbi.nlm.nih.gov/Genbank/index.html [limited by key word search to human sequences]); and the database located at URL http://www.ebi.ac.uk/proteome, among others, are suitable for use in the prescreening methods disclosed herein. In some embodiments, the oligonucleotides identified in the preselection step are subsequently arrayed. In preferred embodiments, the preselected oligonucleotides are entered into the present invention so that the system can generate nucleotide sequences that represent (e.g., complementary) the preselected oligonucleotide sequences.

The present invention is not limited to preselecting oligonucleotide sequences that encode transmembrane proteins. Additional embodiments of the invention provide systems and methods for preselecting oligonucleotides from genomic and other sequence databases based on user specified selection criteria other than their likelihood of encoding a membrane protein. Thus, the present invention provides systems and methods for preselecting oligonucleotide sequences that encode a variety of proteins and polypeptides.

In preferred embodiments, databases of potential transmembrane protein sequences are generated by computational analysis using one or more programs designed to predict transmembrane protein domains. For example, in one embodiment, a total of 282,776 individual protein sequences were assembled in a local sequence database from the above mentioned protein databases. The local sequence database comprised about 30,585 sequences from the Proteome database, about 183,706 from the Ensembl Unconfirmed Database, and about 65,485 sequences from the NCBI nr database, respectively. Since the local sequence database comprises sequences from three (or more) different databases, preferred embodiments of the present invention further contemplate steps to remove sequence redundancies.

The present invention contemplates employing (incorporating) one or more, preferably at least two, transmembrane domain predicting programs, including, but not limited to: 1) maxH (See, Boyd et al., Protein Sci., 7(1):201-205 [1998]); 2) TMHMM (See, Krogh et al., J. Mol. Biol., 305:567-580 [2001]); 3) HMMTOP (See, G. E. Tusnady and I. Simon, Mol. Biol., 283:489-506 [1998]); 4) PREDICTPROTEIN (B. Rost et al., Meth. Enzym., 266:525-539 [1996] [http://cubic.bioc.columbia.edu/predictprotein]); 5) PROF (http://cubic.bioc.columbia.edu/predictprotein); and 6) COILS (A. Lupas et al., Meth. In Enzym., 266:513-525 [1996]) to generate protein sequence databases (MP database) comprising protein sequences likely to provide transmembrane proteins. Using two or more transmembrane domain predicting programs (with different computational methodologies) to predict potential transmembrane domains increases the ratio of actual transmembrane protein sequences to false cell transmembrane protein sequences.

Preferred embodiments of the present invention use (incorporate) the maxH and TMHMM transmembrane protein prediction programs. The present invention is not limited however to using the maxH and TMHMM programs. Indeed, the present invention contemplates using any software program or computation system developed for predicting transmembrane domains in protein sequences (amino acid sequences) or in corresponding nucleic acid sequences. Accordingly, in some embodiments, the MP database further comprises nucleic acid sequences predicted to encode transmembrane protein domains, wherein the nucleic acid sequences are selected by analyzing one or more genomic databases (e.g., GenBank and/or Ensembl Unconfirmed Database) with programs designed to predict gene activity (e.g., GENSCAN, Stanford University, Palo Alto, Calif.). In preferred embodiments, the amino acid sequences derived from the nucleic acid sequences predicted to encode transmembrane proteins are further screened using one or more transmembrane predicting software programs, as described above, before being incorporated into the MP database.

Table 1 shows results from experiments to predict transmembrane proteins from a local sequence database using various combinations of transmembrane predicting software programs.

TABLE 1

| | | Human | Ensembl Predicted |
|---|---|---|---|
| | GenBank nr | Proteome | Unconfirmed |
| | Database | Database | Database |
| TMHMM and | 7925 | 3784 | 7982 |
| maxH positive | | | |
| maxH positive but | 359 | 175 | 6384875 |
| not TMHMM | | | |
| TMHMM positive | 2190 | 1254 | |
| but not maxH | | | |
| positive | | | |

Table 1 shows significant overlap in the transmembrane predicting capabilities of the maxH and TMHMM programs. However, the TMHMM program generated more potential membrane protein candidates. In another experiment, a total of 282,776 protein sequences in the local sequence database were analyzed with the maxH and TMHMM programs of which 29,172 were identified as containing one or more transmembrane domains by either maxH or TMHMM.

In preferred embodiments, the MP database is annotated to comprise additional information about the amino acid sequences predicted to encode transmembrane proteins, including, but not limited to, the number of transmembrane domain(s), and the sequence location of transmembrane domain(s), and the corresponding nucleic acid sequences.

III. Approaches to Microarrays

The ability to attach and probe multiple target molecules (e.g., oligonucleotides or polypeptides) using microarrays provides the capability to simultaneously monitor the expression levels of a large number of genes in many different tissues. Preferred embodiments of the present invention provide systems and methods to rapidly design oligonucleotide sequences that specifically and selectively hybridize to portions of nucleic acid sequences derived from distinct biological samples. In particularly preferred embodiments, the oligonucleotide sequences are arrayed as probes on a microarray for hybridizing to complementary nucleic acids derived from one or more distinct biological samples.

A. Generation of Oligonucleotide Probe Sequences

The present invention provides systems and methods for designing oligonucleotide probes that hybridize to nucleic acid sequences (e.g., gene sequences) derived from one or more distinct biological samples. In preferred embodiments, the present invention provides systems and computer programs for designing one or more unique (e.g., each oligonucleotide probe sequence preferably hybridizes under stringent conditions to a single target nucleic acid in a sample) oligonucleotide probes. The oligonucleotide probe sequences generated by the systems and methods of the present invention thus provide a distinctive “signature” for each nucleic acid of interest entered into the system.

In some embodiments, the system user enters nucleic acid sequence information directly into the system by, for example, by entering the information via a keyboard, computer mouse (i.e., by selecting the sequences from a list or menu), an optical scanner, electronically pasting sequences into a dialogue entry box, or by selecting (based on user set parameters) nucleic acid sequences from one or more sequence databases. The present invention also provides systems and programs that directly receive input from one or more third party software programs designed to search genomic databases for particular nucleic acid sequences of interest (e.g., sequence predicted to encode transmembrane proteins).

Thus, one aspect of the present invention provides users the capability to generate customized in-house oligonucleotide databases wherein each nucleic acid of interest (i.e., a gene or portion thereof) is represented by one (or more) oligonucleotide probe sequence(s). The present invention provides the user the capability to rapidly design and generate unique oligonucleotide probes for use in customized microarrays. The unique oligonucleotide sequences generated by the present invention provide the user with the informational equivalent of an entire genomic database at a fraction of the size. Accordingly, these oligonucleotide databases may be queried for homologous sequences several times faster than full-length DNA sequence databases.

In preferred embodiments, the oligonucleotide probe sequences generated by the systems of the present invention are about 25 . . . 50 . . . 75 . . . 100, or more, nucleotides in length. In particularly preferred embodiments, the oligonucleotide probe sequences are about 50 nucleotides in length. (See, FIG. 1).

In some embodiments, the nucleic acid sequences entered into the system of the present invention comprise one or more genes encoding transmembrane proteins. In preferred embodiments, the nucleotide sequences entered into the system are selected from the sequences that comprise the membrane protein (MP) database described herein. The present invention is not limited however by the nature (e.g., having one or more transmembrane domains) of the nucleic acid sequence entered into systems of the present invention. Indeed, the present invention contemplates the input of nucleic acid sequences encoding a variety of polypeptides and proteins from plants, animals, and microorganisms (e.g., bacteria, archaea, mycoplasmas, viruses, and the like).

In one embodiment, the systems and programs of the present invention generate information comprising: 1) identifying information for nucleic acid sequences entered into the system; 2) the length of the nucleic acid sequences entered in to the system; 3) information regarding the oligonucleotide sequence that hybridizes to a particular nucleic acid sequence entered into the system; and 4) an alignment of the oligonucleotide(s) generated with the nucleic acid entered and a summary of BLASTN results.

In another embodiment, the present invention provides systems comprising: a) providing: i) a user interface capable of receiving user information, wherein said user information comprises one or more nucleic acid sequences, ii) an oligonucleotide sequence generating program operably linked to said user interface, and iii) a computer system having stored therein said oligonucleotide generating program, wherein said computer system comprises computer memory and a computer processor, b) receiving said user information by way of said user interface, and c) processing said user information within said oligonucleotide generating program to generate output (i.e., one or more oligonucleotide sequences that hybridize to a particular nucleic acid sequence entered into the system, and any other additional information generated by system).

For each particular nucleic acid sequence entered into the system by the system user, the present invention generates a list of oligonucleotide sequences (oligonucleotide probe candidates) that have rather high complexity and thus less possibility of forming secondary structures. These oligonucleotide sequences generated are preferred probe candidates for hybridization to the nucleic acid sequences of interest (e.g., when used in microarray-based expression profiling systems). In a preferred embodiment, the oligonucleotide probe candidates are ranked by their proximity to the 3′ end of the nucleic acid sequence (the closer the better) entered and then subsequently ranked again by their (A+T)/(G+C) ratios (the closer to 1 the better). In preferred embodiments the program ensures the uniqueness of the oligonucleotide probe candidates (e.g., a probe candidate hybridizes specifically to only one nucleic acid sequence [gene] entered and preferably that it hybridizes to the 3′ untranslated region [“UTR”]) by automatically comparing all, or a fraction thereof, of the oligonucleotide probe candidates in a particular set against a genomic database (e.g., a human genome database) by running a batch comparison algorithm (e.g., batch-BLAST search) against the database. (See, S. F. Altschul et al., J. Mol. Biol., 215(3):403-410 (1990]). In preferred embodiments, the systems and programs of the present invention use the BLAST algorithm to query the oligonucleotide probe sequences being generated against one or more genome databases (preferably a human genome database, such as, GenBank, ENSEMBLE, GoldenPath, and the like).

In existing methods, the BLAST algorithm is configured to sequentially conduct a comparison of each oligonucleotide in a set of oligonucleotides being analyzed. This limitation consequently makes the process of obtaining the entire BLAST search results for a set of candidate oligonucleotide probes a relatively slow process. However, since only one or a few oligonucleotide probes for a given nucleic acid sequence are typically required to construct a microarray, it is unnecessary to wait for the BLAST algorithm to compare an entire set of oligonucleotide probe candidates against a genomic database.

To address this issue, in preferred embodiments, the present invention provides a “timer” feature that periodically checks the ongoing batch-BLAST results and searches for any oligonucleotides that are unique (as described below) and that fit the user's parameters. The timer feature is incorporated into the present invention as a subroutine (i.e., a subroutine) of the program, or alternatively as a component of system hardware. The timer feature significantly decreases the time required to find high quality oligonucleotide probes that hybridize to a given nucleic (i.e., gene) sequence.

When the desired number of unique oligonucleotide probe candidates for a nucleic acid sequence entered into the system are acquired (e.g., 1, 2, 3, 4, 5 . . . 10 or more), the program stops the BLAST searching process on that particular set of oligonucleotide candidates, and submits a new set of oligonucleotide probe candidates generated for a subsequent nucleic acid (e.g., gene) sequence of interest entered into the system. In particularly preferred embodiments, the BLAST algorithm queries a primary set of oligonucleotide candidates, and parses the results by running one or more keyword/pattern searches. For example, in some embodiments, the BLAST algorithm finds a keyword or pattern (“identity”) in the BLAST results and compares the value of the keyword or pattern to user set parameters (e.g., 100% homology/identity indicates a best hit with 60% maximum tolerance of other hits). The system user may elect to stop the BLAST searching at any point without compromising the quality of the oligonucleotide probe sequences being generated, since the order of oligonucleotides in the BLAST search was previously established by ranking the proximity of the oligonucleotide to the 3′ end of the given nucleic acid sequence (e.g., gene) being analyzed, and by the oligonucleotide's (A+T)/(G+C) ratio.

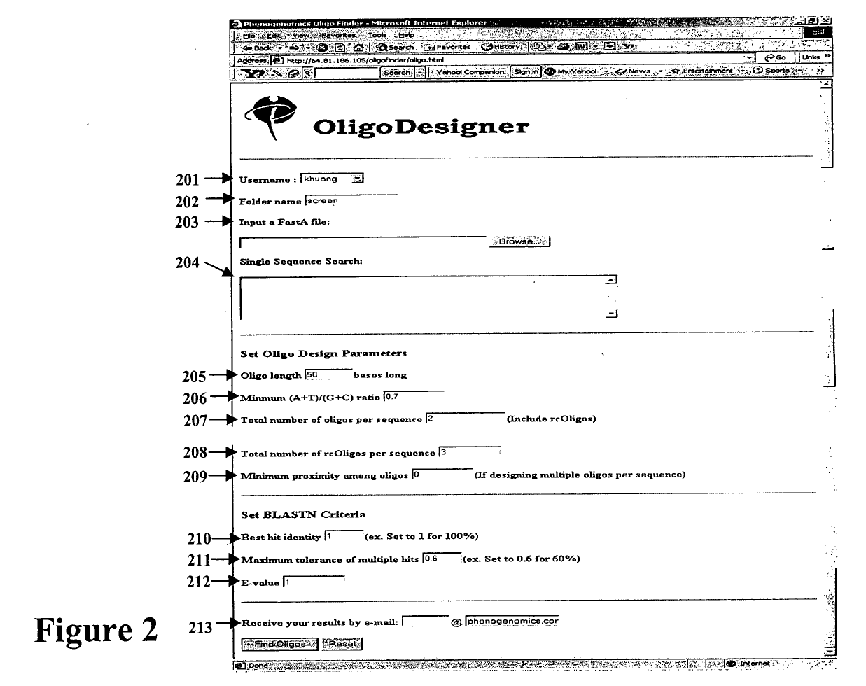

If the system user wants to generate multiple oligonucleotides probe candidates for two or more nucleic acid sequence entered into the system, the system checks the location of all unique oligonucleotides generated, and only reports those unique oligonucleotides that are separated by at least number of bases specified by the system user, thus, when designing/generating multiple oligonucleotides probes per given nucleic acid sequence the system requires the user to preset the desired minimum proximity between oligonucleotides. (See, FIG. 2 at 209 infra).

As mentioned above, the uniqueness of an oligonucleotide probe candidate is based on the BLAST search results. Each oligonucleotide probe candidate generated should have only one 100% sequence homology/identity hybridization partner in a given genome. In particularly preferred embodiments, the BLAST algorithm reports back to the system only those hits (i.e., the hybridization of an oligonucleotide to a sequence in the genome database) that are above the user preset e-value. A given oligonucleotide is only considered to be unique if the BLAST algorithm reports one 100% sequence homology/identity hit between a particular oligonucleotide and the nucleic acid sequence in the genome database being queried. In addition, to be a unique oligonucleotide, the particular oligonucleotide should not have any other hits, other than the one 100% homology/identity hit described above, above the preset acceptance level of sequence homology. For example, if the system user sets the acceptance level at 60%, any additional hits of greater than 60% homology/identity contradicts uniqueness. Selecting unique oligonucleotides for use as probes increases the specificity and signal-to-noise ratio of subsequent hybridization experiments.

In additional embodiments, the systems further comprise devices that receive, process, store, transmit, and display information about the oligonucleotide sequences generated by the system. In certain embodiments, the information output device comprises a monitor. In certain other embodiments, the information output device comprises a printer. In still certain other embodiments, the information output device comprises a means of transmitting the oligonucleotide probe sequences generated by the system to a computer memory device and/or an oligonucleotide-synthesizing device. In preferred embodiments where the information output device transmits oligonucleotide sequence information to an oligonucleotide-synthesizing device, the synthesizing device comprises a column based oligonucleotide synthesizer. The present invention is not intended however to be limited by the nature of the information output device(s).

Other preferred embodiments of the present invention provide the user with a graphic display of the system's information input/out devices and operational status. The present invention contemplates that a graphic display provides the user with the ability to input system operation parameters and to view the results (e.g., oligonucleotide sequences) generated by the system. In some embodiments the system's graphical display comprises a World Wide Web (“Web”) interface (e.g., HTML page) displayed on a computer monitor.

In preferred embodiments, the systems and programs of the present invention are configured to automatically run a version of the NCBI BLASTN algorithm (e.g., BLASTN v2.2.1) to simultaneously screen (analyze) the specificity of multiple oligonucleotides probe candidates generated against the one or more genomic databases (e.g., GenBank). In additional embodiments, the system analyzes all of the oligonucleotide sequences generated to determine their sequence complexity and the existence of secondary structures. The present invention contemplates that each oligonucleotide probe candidate sequence generated will produce only one hit of 100% sequence identity in the given genomic database (e.g., GenBank). If the program detects any other hits above the set e-value, the uniqueness of an oligonucleotide is then determined by the minimum tolerance of the sequence identity of the hits. Thus, the present invention provides a means for significantly reducing cross hybridization.

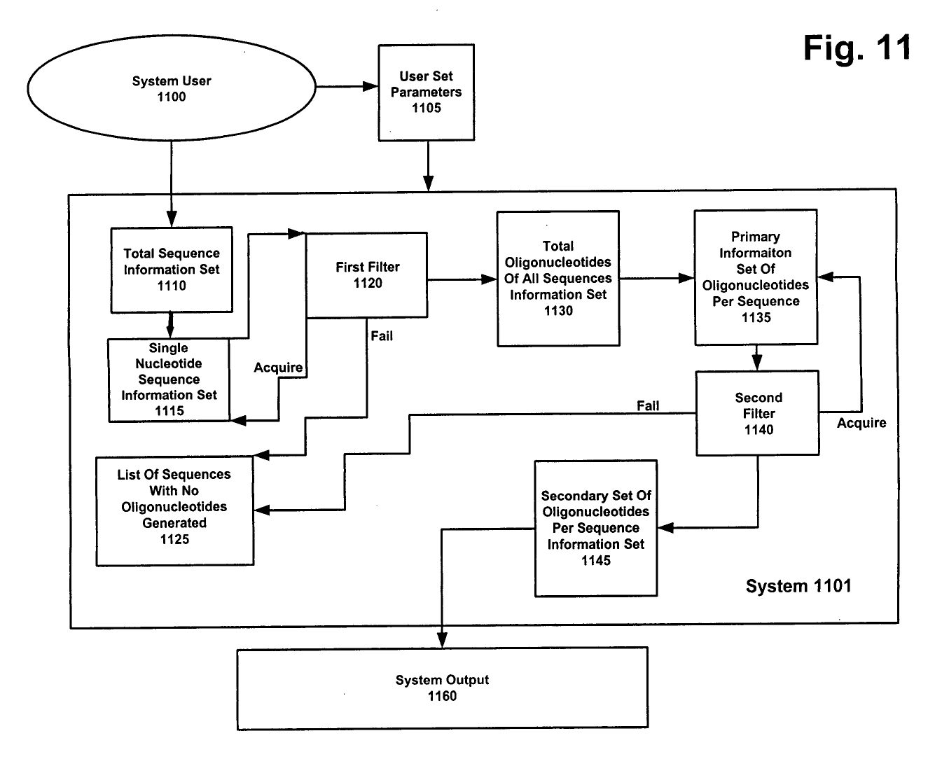

In still further embodiments, the present program provides a built-in timer that directs the program to periodically parse (i.e., analyze) the BLAST results for oligonucleotide specificity. Preferred embodiments of the systems and programs for generating oligonucleotides comprise a processing step referred to as the second filter. (See, FIG. 11 at 1135 infra). In certain of these preferred embodiments, the second filter (1135) comprises two processes. The first process submits a primary oligonucleotide set to a stand-alone, or integrated version, of the BLAST program to query the homology/identity of the oligonucleotides comprising the set against a chosen genomic database (e.g., GenBank). The second process comprises an integrated timer feature that instructs the program to periodically parse (i.e., analyze) the BLAST algorithm's output to confirm that one or more oligonucleotides in a particular set of primary of oligonucleotides is unique. In preferred embodiments, the timer feature of the program activates itself according to user specified time intervals, for example, once every 5 . . . 10 . . . 15 . . . 25 . . . 30 or more seconds. The timer feature preferably rests in an idle mode until it is activated.

In some embodiments, the timer feature comprises an internal clock/counter (i.e., a subroutine within the programs described herein) for determining appropriate activation intervals. In some other embodiments, the clock/counter that instructs the timer feature to activate is remote from main program (e.g., the remote clock/counter communicates with the system and programs of the present invention via a communication link).

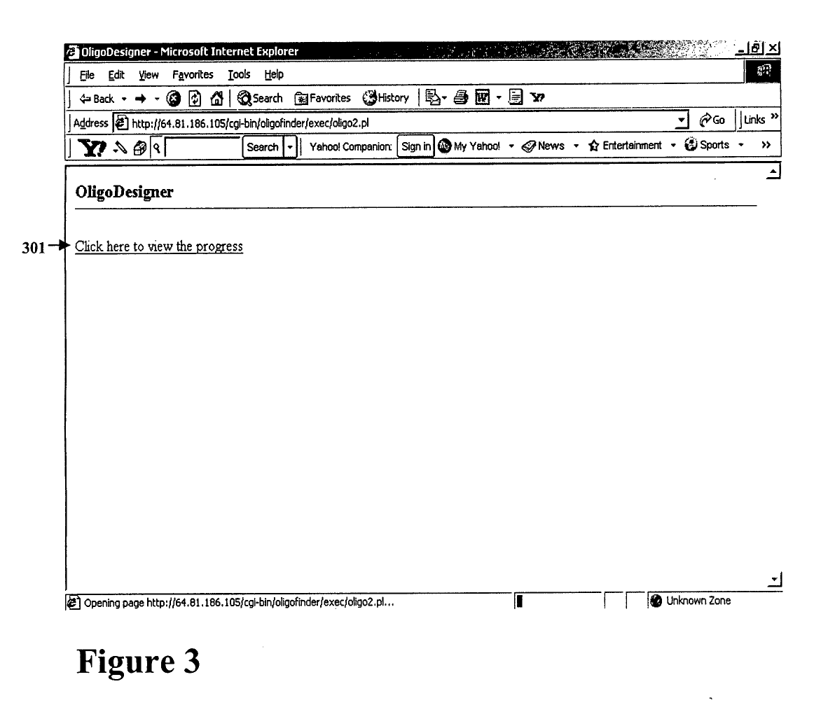

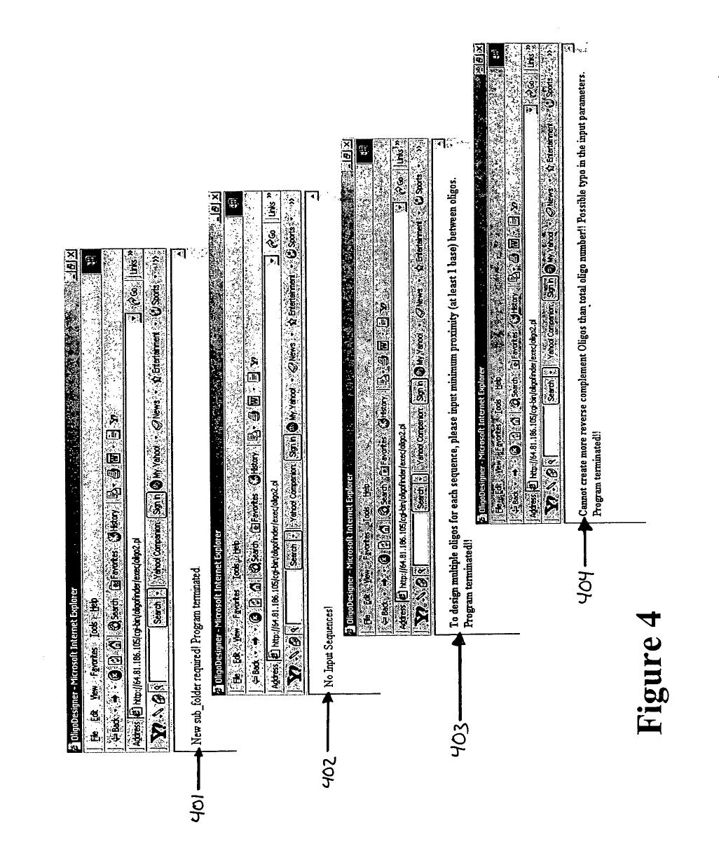

In preferred embodiments, when enough unique oligonucleotides that satisfy the user's requirements (i.e., the user set parameters) for a particular nucleic acid sequence have been acquired, the timer feature terminates the operation of the BLAST algorithm and the main processes of the program are resumed. If not enough unique oligonucleotides have been acquired; the timer goes back into an idle mode and waits for the next instruction (i.e., time period) to parse the BLAST output again. The main processes of the system report the generation/conformation of the unique oligonucleotides for a given nucleic acid sequence to the system user (See, FIGS. 3 and 4 infra) and then repeats the processes of the second filter automatically until all primary oligonucleotide sets have been analyzed and the required number of unique oligonucleotides probe candidates per nucleic acid have been generated, or the system determines that no unique oligonucleotides can be generated for a particular nucleic acid sequence under the current user set parameters.

FIG. 2 through FIG. 11, as described below, exemplify several embodiments of the present oligonucleotide generating systems and programs described herein.

FIG. 2 shows one embodiment of a Web based graphical user (GUI) interface contemplated by the present invention. The systems and programs of the present invention comprise one or more dialogue entry boxes and/or pull down menus (201) that allow the system user to enter (i.e., via a one or more dialogue entry boxes) their system user name and/or other information (e.g., information identifying a particular batch, run, or system session) or to select their user name and other information from lists saved in a computer memory (i.e., via one or more pull down menus). In other embodiments, the system further provides one or more additional dialogue entry boxes (202) that allow the user to specify where system output and other information pertaining to a particular session is to be stored (e.g., in computer memory). In preferred embodiments, the present invention is configured to track system usage and recall particular sessions of by user name or user password, or other session identifiers. (See, FIG. 2 at 201 and 202).

In preferred embodiments, system elements (203) and (204) provide several options for entering nucleic acid sequences and other information into the systems and programs of the present invention. For example, in certain embodiments, system element (203) provides the user with: 1) a dialogue entry box that allows the system user to input the drive path of one or more nucleic sequences already stored in computer memory or a computer readable medium; and/or 2) a pull down menu that similarly allows the user to browse and select from one or more nucleic acid sequences stored in computer memory or available on computer readable medium. In certain embodiments of the systems and programs of the present invention, the user may input from 1, 2, 3, 4, 5 . . . 10 . . . 25 . . . 50 . . . 100 or more nucleic acid sequences (and other information) into the system for analysis (i.e., generating oligonucleotide probes) in a given session or over the course of several sessions. In still other embodiments, the present invention contemplates providing system element (203) for the input of one or more nucleic acid sequence identifiers (e.g., GenBank accession numbers) into the system. In certain of these embodiments, the system is configured to search a local genomic database (e.g., Gen Bank), or to establish a communications link (e.g., Internet connection) with an appropriate remote genomic database to retrieve the specified nucleic acid sequences.

In certain other embodiments, when the user wants to input a single nucleic acid sequence into the system for analysis, the user does so via system element (204), which in preferred embodiments comprises a dialogue entry box for entering the nucleotide sequence of interest (e.g., a nucleic acid sequence in FASTA format).

In other embodiments, the present invention provides the user with one or more options for entering system operational parameters that affect system performance and output, including: 1) dialogue entry box (205) for specifying the desired length (i.e., number of nucleotides) of the oligonucleotide probe candidates being generated; 2) dialogue entry box (206) for specifying the minimum (A+T)/(G+C) ratio in the oligonucleotide probe candidates being generated; 3) dialogue entry box (207) for specifying the total number (e.g., 1.5 . . . 10 . . . 25 . . . 50 or more) of oligonucleotide probe candidates generated per nucleic acid sequence input to the system; 4) dialogue entry box (208) for specifying the number of reverse complement oligonucleotide probe candidates to be generated per nucleic acid sequence input to the system; and 5) dialogue entry box (209) for specifying the minimum proximity (i.e., number of nucleotides) between the first nucleotide of each oligonucleotide probe candidate generated per nucleic acid sequence entered into the system (when the user wants the system to generate more than one oligonucleotide probe candidate per nucleic sequence). Particularly preferred embodiments of the present invention provide each of system elements (205) through (209). The present invention is not intended to be limited, however, to providing system elements (205) through (209). Indeed, the present invention may provide additional dialogue entry boxes, pull down menus, or the like, for entering additional user set parameters and information.

In preferred embodiments, the systems and programs of the present invention generate oligonucleotides that have an (A+T)/(G+C) ratio of about 1. Preferred embodiments of the present invention provide an algorithm that designs oligonucleotides that map to the 3′ end of a nucleic acid sequences entered into the system.

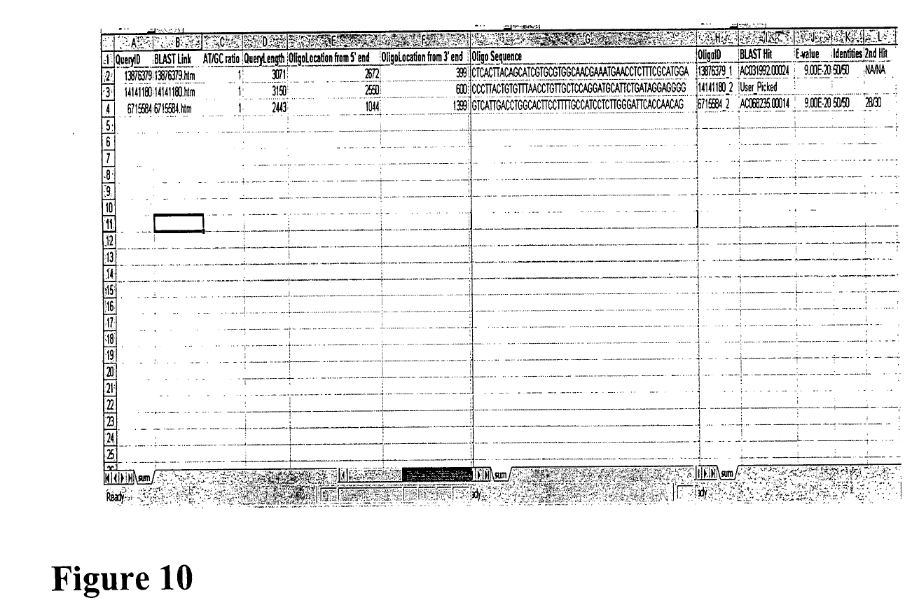

In especially preferred embodiments, the information entered at system elements (205) through (209) comprise the user set parameters that direct the performance of the first filter (See, FIG. 10 at 1005 and 1030 infra).

Various embodiments of the present invention further comprise one or more of system elements (210) through (212). Preferably, the systems and programs employ each of system elements (210) through (212). In certain embodiments, system element (210) provides the user a dialogue entry box for specifying the desired amount of complementarity between the nucleic acid sequence entered into the system and the oligonucleotides (e.g., probe candidates) being generated. For example, entering “1” into dialogue entry box (210) instructs the system to discard any potential oligonucleotides that are not 100% complementary to the desired nucleic acid, while entering “0.9” instructs the system to allow oligonucleotides that are 90% (or greater) complementary to the desired nucleic acid sequence and so on. In certain other embodiments, system element (211) provides a dialogue entry box that allows the user to specify the maximum tolerance between multiple oligonucleotide hits. Because any particular oligonucleotide generated by the system may hybridize with one or more additional genes (or regions of genes) in addition to a particular nucleic acid sequence of interest (i.e., a gene encoded by a particular nucleic acid sequence entered into the system), this parameter allows the user to obtain unique oligonucleotide sequences that that represent the particular gene or sequence of interest.

In preferred embodiments, the system user enters information into system elements (210), (211), and (212) that affects the operation of the system's second filter. (See, FIG. 11 at 1140 infra).

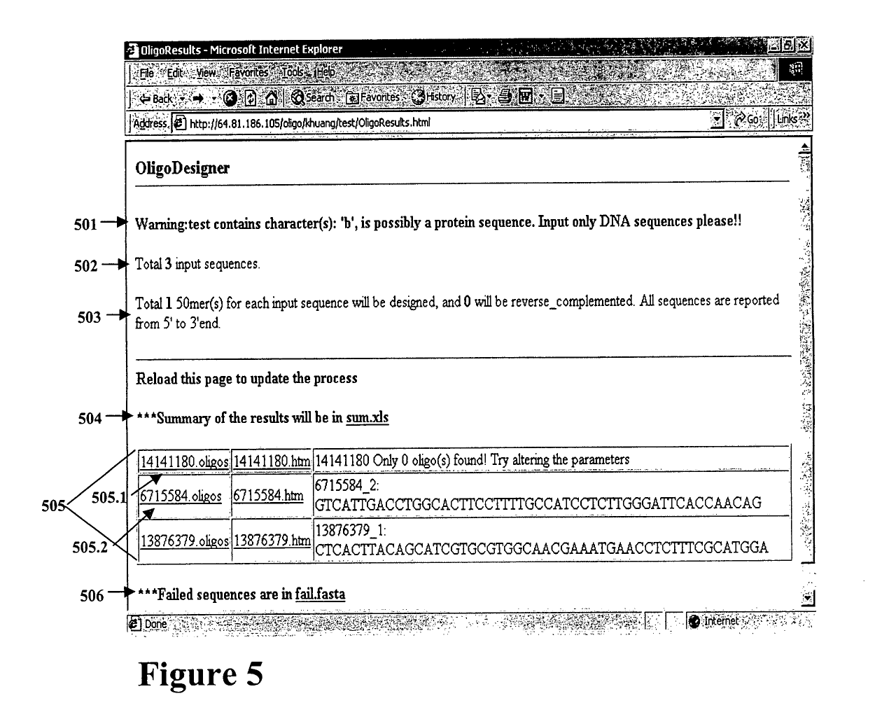

FIG. 3 shows one embodiment of a contemplated Web based GUI of the system after the user has instructed the system to begin processing the nucleic acid sequences entered but prior to displaying system output. In preferred embodiments, the Web based GUI exemplified in FIG. 3 provides a hypertext link (301) to one or more additional Web based GUI screens that display system output in real time as it is being generated. (See, FIG. 5).

In certain embodiments, if the system detects a fatal error in the user set parameters entered into the system, the system displays an appropriate fatal error message while halting system operation thus terminating the session. (See, FIG. 4). For example, in preferred embodiments, the present invention is configured to detect and display a variety of fatal errors occurring in the nucleotide sequences entered and/or the user set parameters, including, but not limited to: 1) an invalid or missing session folder name (See, FIG. 4, 401, “New sub_folder Required! Program terminated.”); 2) no sequences (e.g., nucleic acid sequences) were entered into the system (See, FIG. 4, 402, “No Input Sequences!”); 3) if multiple oligonucleotides per sequence are desired, the minimum proximity among each oligonucleotide probe candidate for the particular sequence has to be specified (See, FIG. 4, 403, “To design multiple oligos for each sequence, please input minimum (at least 1 base) between oligo. Program terminated!”); 4) error where the number of reverse oligonucleotide probe candidates desired is higher than the total number of oligonucleotide probe candidates desired (See, FIG. 4, 404, Cannot create more reverse complement oligos than total oligo number! Possible typo in the input parameters. Program terminated!”); 5) an invalid or missing user name (“No given user name”); and 6) the nucleic acid sequences entered into the system are shorter than the desired oligonucleotide probe candidates. In preferred embodiments, the user can print a description of any (fatal) error messages that might arise or of any other information, including system output.

FIG. 5 shows one embodiment of a contemplated Web based GUI that displays system output in real time as it is being generated. Accordingly, in certain embodiments, the system is configured to allow the user to periodically reload the GUI depicted in FIG. 5 to update the display of system performance and the generated being generated. In other embodiments, the display updates itself automatically. This feature allows the user to determine how many nucleic acid sequences have been processed and how many sequences are remaining. In other embodiments, the Web based GUI depicted in FIG. 5 is configured to display the total system output (or a portion thereof) at the completion of the session.

In reference to FIG. 5, in some embodiments, the present invention provides system element (501) that warns the user of one or more nonfatal problems that could prevent oligonucleotides (probe candidates) from being generated for a particular nucleic acid sequence entered into the system. For example, in some embodiments, the system displays a warning message alerting the user that one or more nucleic acid sequences entered into the system contains characters inconsistent with FASTA nucleic acid sequence coding (e.g., a sequence contains one or more characters designating an amino acid). Accordingly, in preferred embodiments, the system provides a subroutine that scans each nucleic acid sequence entered by the user for incongruities with FASTA nucleic acid sequence format. In some embodiments, the system is configured to report any sequence format errors and then continue processing the remainder of the proper nucleic acid sequences entered by the user. In other embodiments, the system is configured to report the error and stop processing sequences until the user corrects the error (e.g., deletes the offending sequence) or otherwise instructs the system to resume processing thus overriding the error message.

In some preferred embodiments, system elements (502) and (503) provide the user with a synopsis of the number of nucleic acid sequence entered into the system for processing, the number of oligonucleotide probe candidates (or other sequences such as PCR primers) that will be generated per nucleic acid sequence entered, the length of the oligonucleotides (in nucleotide bases), and the number of desired reverse complement sequence per nucleic acid sequence entered, if any. Referring to the exemplary GUI depicted in FIG. 5, the user instructed the system to generate three 50 base oligonucleotides that hybridize one of each of the three nucleic acid sequences entered, and that no reverse complement sequences are desired. (See FIG. 5 at 502 and 503). In still other embodiments, additional system information or instructions entered by the user are displayed by system element (503).

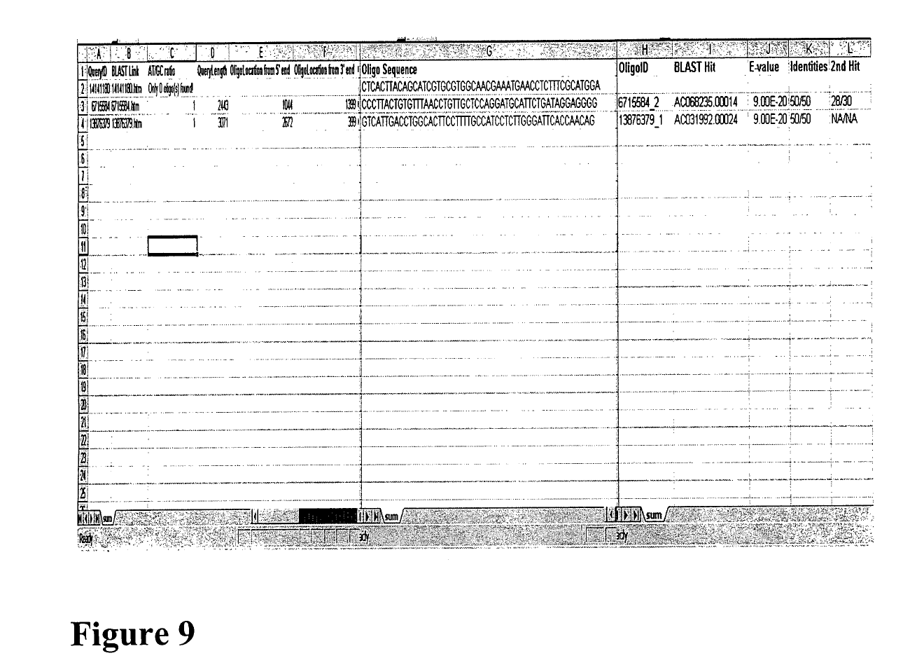

As mentioned above, in certain embodiments, the system provides the user a display of system output in spreadsheet format (e.g., Microsoft Excel). In certain embodiments, the present invention provides a hypertext link (504) to real time system output in spreadsheet format. (See, FIG. 9). In other embodiments, system output (e.g., the oligonucleotide sequences generated) is displayed in plain text format that is universally compatible with any GUI based (e.g., Windows) program.

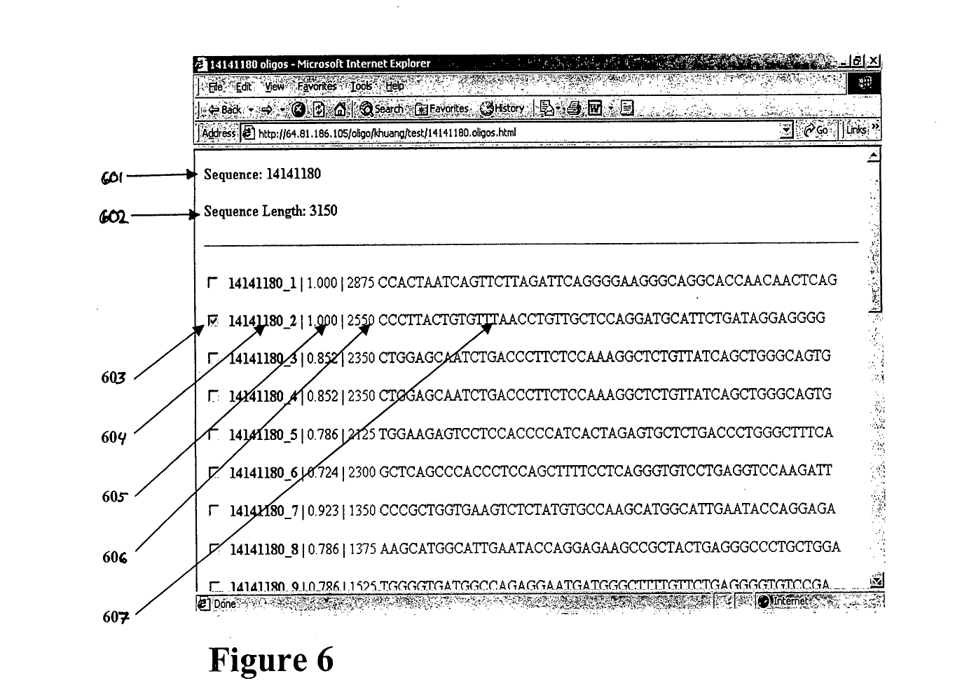

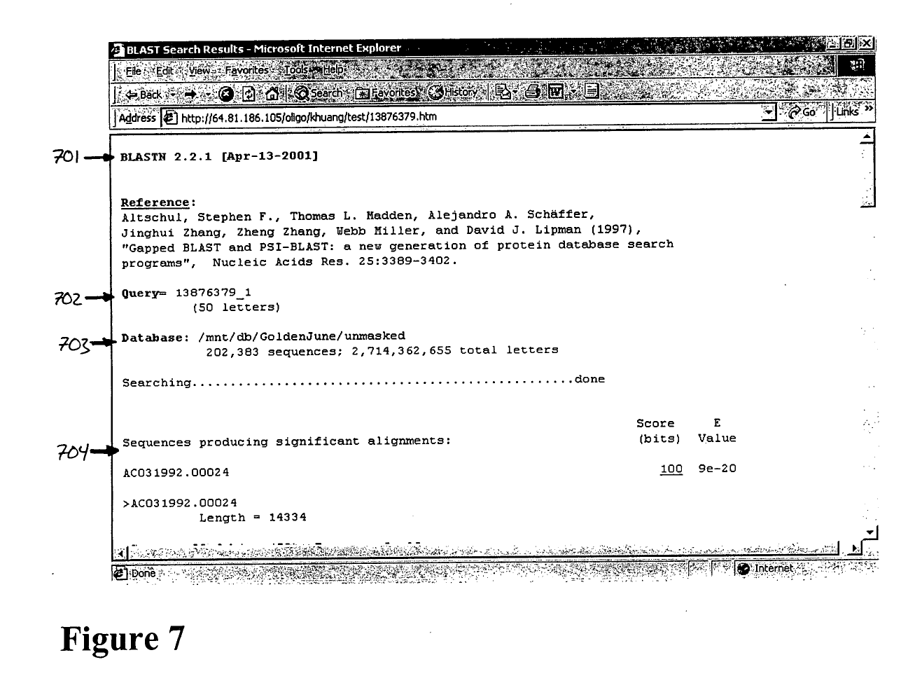

Depending on the total number of nucleic acid sequences entered into the system for analysis in a given session, the GUI contemplated in FIG. 5 provides from 1-100, preferably from 1-50, more preferably from 1-25, and most preferably from 1-10 lines of information presenting system output. (See, FIG. 5 at 505). In particularly preferred embodiments, each display line (shown at 505) corresponds to the system's output for an individual nucleic acid sequence entered into the system. Preferably, the user is able to scroll through various display lines. For example, system element (505) shows 3 display lines, each line corresponding to the system's output (i.e., results) for a particular nucleic acid sequence entered into the system. In particular, line (505.1) shows that the system failed to generate an oligonucleotide that hybridizes to nucleic acid sequence 14141180 (under the parameters specified by the system user). Referring to line (505.1), the reader will notice three sections when reading from left to right. Section 1 (the left most section) of line (505.1) provides a hypertext link to a FASTA file comprising the primary set oligonucleotides generated by the system for a particular nucleic acid sequence. (See, FIG. 6). Section 2 (the middle section) of line (505.1) provides a hypertext link to a .html file comprising the raw BLAST search data for a particular nucleic acid sequence. (See, FIG. 7). Section 3 (the rightmost section) of line (505.1) alters the user to the failure of the system to generate an appropriate oligonucleotide under the current user set parameters. Line (505.2), however, shows that the system successful generated an oligonucleotide that hybridizes to nucleic acid sequence 6715584 (under the user specified parameters). Referring to section 3 (the rightmost section) of line (505.2) the top portion of this section identifies the number of the oligonucleotide generated by the system that best meets user set parameters (e.g., oligonucleotide 6715584_{—}2 shows that the second oligonucleotide generated by the system upon processing nucleic acid sequence 6715584 best meets the user's parameters for oligonucleotides generated by the system). The bottom portion of this provides the FASTA nucleic acid sequence of the sequence that best meets the user's parameters for the given nucleic acid sequence entered (e.g., the sequence of nucleic acid 6715584_{—}2).

In some embodiments, the system provides a hypertext link (506) to a file comprising the nucleic acid sequences for which the system failed to generate oligonucleotides (probe candidates) under the user set parameters. In certain of these embodiments, this file is displayed in FASTA format. In particularly preferred embodiments, the system provides a hypertext link (or checkbox) that corresponds to each sequence that failed to generate an acceptable oligonucleotide that redirects the system user to a GUI (e.g., the depicted in FIG. 5) thus allowing the user to quickly alter the user set parameters and reprocess the failed sequence(s). (See, FIG. 6). The present invention contemplates that this feature will decrease total session processing times.

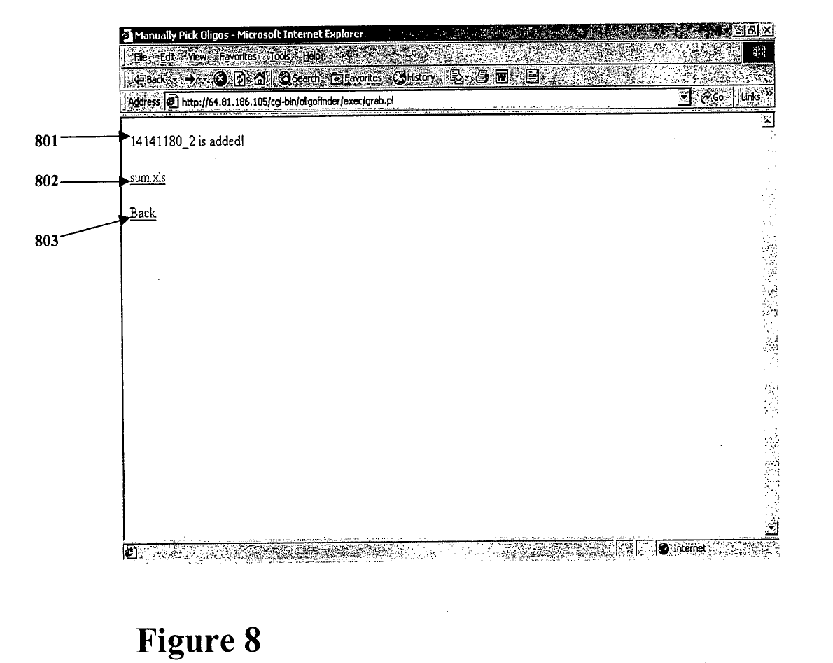

FIG. 6 shows one contemplated Web based GUI showing a primary list of oligonucleotides generated for a particular nucleic acid sequence entered into the system. (See, FIG. 5, line 505.1, leftmost section). FIG. 6 shows the first eight oligonucleotides generated corresponding to nucleic acid sequence 14141180 entered by the system user into the system. Briefly, system feature (601) identifies the nucleic acid sequence being analyzed. System feature (602) provides the number of nucleotides in the nucleic acid sequence. In some embodiments, a checkbox (603), is provided for each primary oligonucleotide displayed. In certain of these embodiments, if the system user checks (e.g., clicks a checkbox using a mouse) one or more of these checkboxes, the system is instructed to include the corresponding primary oligonucleotide sequences with the system output. (See, FIGS. 8 and 10). In preferred embodiments, the checkbox (603) can be used to select one or more primary oligonucleotides that correspond to nucleic acid sequences entered into the system that failed to generate unique oligonucleotides.

System feature (604) provides the identity of the primary oligonucleotide displayed in each line (e.g., 14141180_{—}2 identifies the second oligonucleotide generated by the system that corresponds to nucleic acid sequence 14141180 entered by the user). System feature (605) displays the (A+T)/(G+C) ratio of the identified oligonucleotide (e.g., primary oligonucleotide 14141180_{—}2 has an (A+T)/(G+C) ratio of 1.00). System feature (606) displays the location of the particular primary oligonucleotide from the 5′ end of the nucleic sequence from which it was generated (e.g., the first base of oligonucleotide sequence 14141180_{—}2 is 2550 bases from the 5′ end of nucleic acid sequence 14141180). System feature (607) displays the nucleic acid sequence of the particular primary oligonucleotide being represented.

FIG. 7 shows one contemplated Web based GUI display showing the raw BLASTN results generated for one particular nucleic acid sequence entered into the system. System feature (701) identifies the version of the BLAST program being run. System feature (702) identifies the nucleic acid sequence being analyzed and the number of nucleotides in the sequence. System feature (703) identifies the database queried by the BLASTN program. System feature (704) displays BLAST search results generated by the system.

FIG. 8 shows one contemplated Web based GUI confirming that the user has chosen to manually add a primary oligonucleotide sequence to the system's output. For example, in certain of these embodiments, system element (801) identifies primary oligonucleotides the user has chosen to add to the system's output (e.g., the user has chosen to add primary oligonucleotide 14141180_{—}2; See, FIG. 6 at (601)). In certain other of these embodiments, system element (802) provides a hypertext link to an updated spreadsheet (e.g., Microsoft Excel) display of system output, including any primary oligonucleotide sequences the user has manually chosen to add to the system's output. (See, FIG. 10). In yet still other embodiments, system element (803) provides a hypertext link that directs the user back to the primary list of oligonucleotides generated for a particular nucleic acid sequence entered into the system (See, FIG. 6).

FIG. 9 shows one contemplated GUI for displaying system results and other information when the user selects to view system output in spreadsheet format (e.g., Microsoft Excel). Briefly, in some embodiments, the spreadsheet provided by the system comprises a number of columns for displaying information about sequences being analyzed and system output. For example, FIG. 9, column (A) identifies the nucleic acid sequence processed by the system. FIG. 9, column (B) provides a hypertext link to the BLAST results generated by the system (e.g., a hypertext link to the information displayed in FIG. 6 and/or FIG. 7). In other embodiments, FIG. 9, column (C) provides the (A+T)/(G+C) ratio of the particular nucleic acid sequence generated. FIG. 9, column (D) provides the length (e.g., number of nucleotides) of the particular nucleic acid sequence being analyzed by the system. In still other embodiments, FIG. 9, column (E) provides the distance from the '5 end of the nucleic acid sequence entered into the system of the oligonucleotide generated. Conversely, in some embodiments, FIG. 9, column (F) provides the distance of the oligonucleotide generated by the system from the 3′ end of the nucleic acid sequence entered. FIG. 9, column (G), provides the sequences of the oligonucleotide generated by the system. In preferred embodiments, one column (e.g., FIG. 9 column (H)) provides the unique identifier of the oligonucleotide generated by the system that best meets the user's requirements for a particular nucleic acid sequence entered into the system. FIG. 9, column (I), provides BLAST hit information for a particular oligonucleotide generated by the system. In still other embodiments, FIG. 9, columns (J), (K), and (L), provide the e-value, ratios of sequence identities, and 2nd hit values, respectively, for a particular oligonucleotide generated by the systems and programs of the present invention as described herein.

The information displayed in the model spreadsheet shown in FIG. 9, is not intended to be limited to what is described in the columns noted above, nor are the columns noted above required to appear in the order provided. Indeed, the present systems and programs may be configured to display a variety of information one skilled in the art would consider pertinent to the operation of the systems and programs disclosed herein.

FIG. 10, shows one contemplated GUI spreadsheet format (e.g., Microsoft Excel) for displaying system output after the manual addition of one or more primary oligonucleotide sequences, for example, primary oligonucleotide 14141180_{—}2. (See, FIG. 10, column (I)). The columns depicted in the model spreadsheet shown in FIG. 10, are explained above in the description of FIG. 9.

FIG. 11 shows a diagrammatic representation of one contemplated embodiment of the systems and programs of the present invention stored in computer readable memory implemented (run) on a computer processor. In preferred embodiments, various system elements, as described herein, are operably linked such that information entered into the system or being generated by the system is readily exchanged between system elements. For example, the User Set Parameters 1105, described below, are operably linked to System 1101. In another example, the Total Sequence Information Set 1110, described below, is operably linked to the Single Nucleotide Sequence Information Set 1115. It will be apparent to one skilled in the art that multiple linkages are contemplated throughout the systems and programs of the present invention.

Briefly, the System User (1100) enters into the System (1101) various User Set Parameters (1105) that affect the characteristics of the oligonucleotides (probe candidates) generated by the System (1101). (See, FIG. 2 at 201-211). As described above, in some embodiments, the System User (1100) enters information (i.e., User Set Parameters (1105)) into the System (1101) regarding the desired: 1) desired length of the oligonucleotides generated; 2) the number of oligonucleotide generated per nucleic acid sequence entered into the system; 3) the minimum (A+T)/(G+C) ratio of the oligonucleotides generated; 4) the minimum proximity among multiple oligonucleotides generated; and 5) the System User (1100) may elect to have the System (1101) remove oligonucleotides generated that contain simple repeats (e.g., homoploymers, di-, or tri-nucleotide repeats, and direct or inverted repeats). The System User (1100), however, is not limited to entering information into the System (1101) concerning only the above-mentioned User Set Parameters (1105). In preferred embodiments, the System User (1100) enters User Set Parameters (1105) into the System (1101) through a GUI, or one or more dialogue entry boxes.

The System User (1100) enters the Total Sequence Information Set (1110) to be analyzed into the System (1101) (e.g., through a GUI or, dialogue entry box, or any other method/hardware device). In preferred embodiments, the System (1101) acquires single nucleotide sequences one at a time (e.g., the Single Nucleotide Sequence Information Set (1115)) to analyze in the First Filter (1120) from the Total Sequence Information Set (1110) entered. In preferred embodiments, the First Filter (1120) sequentially analyzes the complexity and likelihood of secondary structure in each of the sequences comprising the Total Sequence Information Set (1110). Sequences analyzed by the First Filter (1120) having high sequence complexity and/or low possibility for secondary structures are candidates for further processing by the System (1101). In preferred embodiments, nucleic acid sequences with low sequence complexity (e.g., regions of biased composition including, but not limited to, homopolymeric runs, short-period repeats, and subtle over representation of one or more residues) and/or a high probability of secondary structure are withdrawn from further analysis and diverted by the System (1101) into the List Of Sequences With No Oligonucleotides Generated (1125) stored in computer readable memory. In certain of these embodiments, the present invention employs the DUST program as a subroutine to mask or filter low complexity regions in the nucleic acid queried.

In preferred embodiments, the First Filter (1120) creates a set (2 . . . 5 . . . 10 . . . 50 . . . 100 or more) of individual oligonucleotides (25 . . . 50 . . . 75 . . . 100 nucleotides or more in length) that correspond to a particular nucleic acid sequence entered into the System (1101) comprising the Total Sequence Information Set (1110). In preferred embodiments, the First Filter (1120) takes each nucleic acid sequence entered into the System (1101) (orientated from 5′ to 3′) and breaks each particular nucleic sequence into variable units. For example, if the System User (1100) wants the system to generate 50 nucleotide long oligonucleotides then a unit would comprise about 300 bases. In preferred embodiments, the program designates one of these units as a terminal unit (e.g., the 300 bases at the 3′ end of the given sequence) identifying this unit as unit number and subsequently moving in the 5′ direction along the nucleic acid sequence 250 bases at a time from the first base of the first unit (terminal unit) to arrive at the beginning of the second unit, and so on, until the entire nucleic acid sequence is divided into a number of 300 base long units. Accordingly, in preferred embodiments, the systems and programs of the present invention contemplate creating a series of interlocking units that overlap at their 3′ ends (in the example presented adjacent units have a 50 base overlap). In preferred embodiments, the programs and systems of the present invention rank the units according to their proximity to the 3′ end (the closer the higher the ranking) of particular nucleic acid sequence being analyzed. In some embodiments, each unit is in turn further divided into shorter subunits (e.g., fragments) based on the size of the desired oligonucleotides to be generated (e.g., if the user wants to generate 50 base long oligonucleotides, the program starts from 5′ end of the first designated subunit and acquires successive 50 base fragments starting at the first base of the subunit (or at any other base), and then acquires a second, third, and fourth, etc., 50 base long fragment downstream of that starting point by shifting some multiple of three bases (i.e., the program indexes one or more reading frames towards the 3′ end of the subunit), thus, a series of nested (i.e., overlapping) 50 base long fragments are generated from each subunit. In preferred embodiments, the set of oligonucleotides generated by the First Filter (1120) comprise a set of “nested” oligonucleotides (e.g., an oligonucleotide within the nested set shares an overlapping region of identity with one or more other oligonucleotides in the set).

In particularly preferred embodiments, the set of nested oligonucleotides is generated by, and corresponds to, successive three (or any multiple of three, e.g., 3, 6, 9, etc.) nucleotide reading frame shifts along a particular nucleotide comprising the Single Nucleotide Sequence Information Set (1115). In preferred embodiments, the oligonucleotides comprising the nested set are each about 50 bases.

In other preferred embodiments, fragments from the same subunit are not ranked by their location, in other words, the fragments are treated as equal in terms of location ranking. However, as mentioned above, in preferred embodiments the original units were already ranked by their proximity to the 3′ end of the given nucleic acid sequence.

In preferred embodiments, the program sequentially checks the sequence complexity of all fragments in each unit/subunit. In certain of these embodiments, all fragments generated from a particular unit/subunit are filtered by the system for sequence repeats including, but not limited to: single nucleotide repeats (e.g., AAAAA, GGGGG), di-nucleotide repeats (e.g., ATATAT, GAGAGA), tri-nucleotide repeats (e.g., ATGATGATG, GCTGCTGCT), sequence repeats (e.g., AATGAATG, AATGNNNAATG), palindromes (e.g., AATGCATT, AATGNNNCATT). In still other embodiment, the program then filters (e.g., discards from further analysis) those fragments having the above-mentioned patterns (e.g., single, di-, and tri-nucleotide repeats, sequence repeats, and palindromes), such that the remaining fragments (oligonucleotide probe candidates) are then ranked by (A+T)/(G+C) ratio. In preferred embodiments, (A+T)/(G+C) ratios closer to 1 are better oligonucleotide probe candidates. (A+T) means the total count of As and Ts in the fragment and (G+C) means the total count of Gs and Cs. To determine how close the (A+T)/(G+C) ratio is to 1, the program calculates (ratio −1)₂, the smaller the value of this formula the closer the ratio is to 1. In preferred embodiments, the primary set of oligonucleotide candidates are then stored in computer readable memory based on their rank, first, by the proximity of the unit from the 3′ end of the given nucleic acid sequence, and second, oligonucleotide probe candidates from the same subunit are then again ranked based on their (A+T)/(G+C) ratios. The present invention considers fragments from the same unit as being equivalent in terms of their sequence location because in hybridization experiments, oligonucleotides from the same local region do not affect hybridization significantly. However, the reason that fragments from the same unit are ranked based on their (A+T)/(G+C) ratios, is because the ratios have a direct affect on hybridization conditions. When comparing oligonucleotides generated from the same 300 nucleotide long units, the (A+T)/(G+C) ratio is a more important factor than sequence location, because most cDNAs are generated from their 3′ end, are often not full length, and their 5′ end may be missing.

In preferred embodiments, System (1101) conducts a stepwise analysis of each nucleic acid sequence (e.g., each nucleic acid sequence comprising the Single Nucleotide Sequence Information Set (1115)) until each sequence has been analyzed by the First Filter (1120) and a set of nested oligonucleotides has been generated for each nucleic acid entered, as described above, or until an aggregate Total Oligonucleotides Of All Sequences Information Set (1130) has been created. In still other embodiments, System (1101) is configured to bypass creating the Total Oligonucleotides Of All Sequences Information Set (1130) from the sequences created by the First Filter (1120) to transfer the sequences (i.e., the nested set of oligonucleotides) to downstream System (1101) databases/filters (e.g., 1135, 1140, etc.). In preferred embodiments, the Total Oligonucleotides Of All Sequences Information Set (1130) comprises an aggregate of all the nested sets of oligonucleotides created from each of the Single Nucleotide Sequence Information Set (1115) entered. In some embodiments, the Total Oligonucleotides Of All Sequences Information Set (1130) is stored in a computer readable memory device. In still other embodiments, the System (1101) sequentially selects one set of nested oligonucleotides from the aggregate of nested oligonucleotides sets comprising the Total Oligonucleotides Of All Sequences Information Set (1130) for further analysis by the Second Filter (1140). Each Primary Oligonucleotides Per Sequence Information Set (1135) corresponds to and represents one Single Nucleotide Sequence Information Set (1115) selected from the Total Sequence Information Set (1110).

In preferred embodiments, the Second Filter (1140) further analyzes the sets of nested oligonucleotides that comprise the Primary Oligonucleotides Per Sequence Information Set (1135) by screening (i.e., analyzing) the specificity of each individual oligonucleotide within a particular set against the sequences contained in one or more genomic databases (e.g., GenBank). In preferred embodiments, the System (1101) and the Second Filter (1140) accomplish this step by running a local version of the NCBI BLASTN program. In other embodiments, the System (1101) establishes a communications link (e.g., an Internet connection) with a remote version of the NCBI BLASTN program to accomplish this step. In preferred embodiments, as mentioned above, System (1101) uses a timer feature (5 . . . 10 . . . 15 . . . 45 sec., etc.) to limit the number of comparisons run in the NCBI BLASTN program per Primary Oligonucleotides Per Sequence Information Set (1135) and/or per each individual oligonucleotide comprising the Primary Oligonucleotides Per Sequence Information Set (1135). Similar in the operation of the First Filter (1120), the Second Filter (1140) sequentially selects one set of nested oligonucleotides at a time for further analysis from the Primary Oligonucleotides Per Sequence Information Set (1135). In preferred embodiments, the Second Filter (1140) is configured to reject individual oligonucleotides comprising the Primary Oligonucleotides Per Sequence Information Set (1135) that do not meet the User Set Parameters (1105). The oligonucleotides rejected by the Second Filter (1140) are transferred to the List Of Sequences With No Oligonucleotides Generated (1125) stored in computer readable memory. In preferred embodiments, the Second Filter (1140) analyzes individual oligonucleotides based on: 1) uniqueness in the genome database; 2) rank based on proximity to the 3′ end of the Single Nucleotide Sequence Information Set (1115) entered; and 3) rank (A+T)/(G+C) ratio, although the System (1101) is not intended to be limited by the particular User Set Parameters (1105) entered into the System (1101).

The oligonucleotides sequences comprising the Primary Oligonucleotides Per Sequence Information Set (1135) that met the User Set Parameters (1105) and pass the Second Filter (1140) are stored in computer readable memory by the System (1101) as the Secondary Set Of Oligonucleotides Per Sequence Information Set (1145).

In preferred embodiments, oligonucleotide sequences output by the systems and programs of the present invention are transferred (e.g. via communications link) to one or more oligonucleotide synthesizing machines. Accordingly, in preferred embodiments, oligonucleotide molecules are synthesized based on the oligonucleotide sequences generated by the System (1101) and subsequently arrayed on a substrate (e.g., a solid substrate). The art is familiar with the nature and operation of automated oligonucleotide synthesizing machines and oligonucleotide arrays.

B. Advantages of the Present Systems and Programs

The systems and methods of the present invention for generating sequence databases, oligonucleotide (probe) sequences, and subsequent arrays provide several advantages over existing gene expression profiling methodologies. For example, the systems and methods disclosed herein are useful for rapidly identifying differentially-expressed cell membrane proteins as potential targets for antibody based therapeutics. Antibody based drug discovery methods provide several advantages over pharmacological drug discovery processes that focus on designing and testing lead compounds. Several additional advantages of the present invention are listed below:
1. The systems and methods of present invention provide a comprehensive data set for gene expression analysis since, in one embodiment, the entire human genome sequence is used as starting material;
2. The systems and methods of present invention provide, in one embodiment, the entire repertoire of cell membrane proteins for expression profiling;
3. The systems and methods of present invention provide smaller differential display data sets since about 70% of the genes that cannot be recognized by antibodies (i.e., genes that do not encode cell membrane proteins) are eliminated prior to display;
4. The systems and methods of present invention provide higher throughput rates when identifying cell membrane protein targets than traditional approaches;
6. The systems and methods of present invention provide an approach for rapidly identifying and screening cell membrane protein targets for a particular disease than traditional approaches; and
7. The systems and methods of present invention are not limited, however, to identifying targets for antibody development since some of the genes encoding differential expressed cell membrane proteins will also be targets for chemical therapeutics.

Additional advantages of the systems and methods of the present invention will be apparent to those skilled in the art.

C. Preferred Microarray Methods

Microarrays provide powerful tools for analyzing numerous types of biomolecules, as well as other organic and inorganic samples. Microarray techniques have been developed for comparative studies of DNA, RNA, and mRNA from a number of organisms (e.g., vertebrates, invertebrate, plants, yeasts, fungi, bacteria, viruses, etc.). Some microarray applications use the “polymerase chain reaction” (PCR) for generating sufficient quantities of probe sequences. Alternatively, nucleic acid synthesis methods can be used for generating shorter and precise oligonucleotide probe sequences. Microarrays can also be fabricated with purified or synthetic polypeptides probes. Similarly, whole cells or fragments of cells can be assayed in microarray format.

In preferred embodiments, association with one or more solid or semi-solid substrates supports the microarray elements of the present invention. Array substrates may comprise planar (i.e., 2 dimensional) glass, metal, composite, or plastic slides and wafers, biocompatible or biologically unreactive compositions, and porous or structured (i.e., 3 dimensional) substrates of the same, or similar, composition as those utilized in 2 dimensional substrates. For example, common planar arrays include 1 in.×3 in. microscope slides (1×25×76 mm) and yield approximately 19 cm2 of surface area (enough surface area for >100,000 array features using current microspotting and ink-jetting arraying technologies). Currently microscope slides are being manufactured using ultraflat substrates (ultraflat, also known as “optically flat,”) surfaces that help to eliminate data acquisition errors resulting from out of focus array elements on uneven substrate surfaces) (TeleChem, Sunnyvale, Calif.). Specially manufactured, or chemically derivitized, low background fluorescence substrates (e.g., glass slides) are also commercially available. In yet other embodiments, planar microarray substrates further comprise cover slips, gaskets, or other enclosures that protect the array elements and provide channels for the flow of chemical and reagents for microarray preparation, hybridization, labeling, etc. Microarray elements may be prepared and analyzed on either the top or bottom surface of the planar substrate (i.e., relative to the orientation of the substrate in the data acquisition device of the present invention).

Those skilled in the art well appreciate that certain substrate preparation steps may be necessary in order to prepare the chosen substrate for receiving microarray element features. For example, glass or plastic substrate slides are often treated under harsh conditions with strong acids or detergents to remove undesired organic compounds and lipids prior to association with microarray probe features (e.g., nucleic acid sequences).

In some embodiments, the microarray substrates (e.g., glass slides) are associated or derivitized with one or more coatings and/or films that increase microarray element probe-to-substrate binding affinity. Increased microarray probe binding to substrates leads to increased microarray probe retention during the various stages of microarray preparation and analysis (e.g., hybridization, staining, washing, scanning stages, and the like, of microarray preparation and analysis).

Numerous techniques for associating microarray elements with microarray substrates exist. For example, microarray elements may be located on suitable substrates by non-contact or contact systems. Non-contact systems typically comprise ink-jet like, or piezoelectric printing technologies. Contact based microarray printing technologies utilize slender pins, with or without fluid retaining grooves and wells, involve “tapping” the printing pins directly onto the surface of the microarray substrate.

In some preferred embodiments of the present invention, the microarrays are fabricated using photolithographic technologies. For example, U.S. Pat. Nos. 5,744,305, 5,753,788, and 5,770,456 (herein incorporated by reference in their entireties) describe photolithographic techniques for directly fabricating microarray elements on a rigid substrate using photolabile protecting groups and a number of fixed-pattern light masks for selectively deprotecting array elements for nucleoside concatenation at each base addition step. A “maskless” microarray fabrication technology is also known (See e.g., WO/9942813). In a preferred embodiment, the present invention can be used to acquire data sets from microarrays fabricated utilizing the maskless array fabrication technology disclosed in WO/9942813. In another embodiment, microarrays are fabricated in a manner, in whole, or in part, similar to that described in WO/9942813 by the system of the present invention, and then “read” (i.e., data is acquired from the microarray) by the system of the present invention.

In embodiments where nucleic acids (e.g., DNA) comprise the probe elements, a hybridization step is typically carried out to bind a target, either labeled or unlabeled, to the probe elements. The particular hybridization reaction conditions can be controlled to alter hybridization (e.g., increase or decrease oligonucleotide binding stringency). For example, reaction temperature, concentrations of anions and cations, addition of detergents, and the like, can all alter the hybridization characteristics of microarray probe and target molecules.

To generate data from microarray assays a signal is detected that signifies the presence of, or absence of, the sequence of, or the quantity of the assayed compound or event. In preferred embodiments, the signal involves a measurement of fluorescence. Briefly, fluorescence occurs when light is absorbed from an external (excitation) source by a fluorescent molecule (a fluorophore) and subsequently emitted. The emitted light is of a lower energy (longer wavelength) than the absorbed light because some of the excitation energy is dissipated upon absorption. The characteristic spectral shift between excitation and emission wavelengths of a particular fluorophore is called the Stokes shift. Discrimination between excitation wavelengths and emission wavelengths improves the signal to noise ratio and dynamic range of the detector system by substantially removing background fluorescence and scattered excitation light from fluorophore-specific emission.

In embodiments where the microarray comprises nucleic acids, the present invention further contemplates direct and indirect labeling techniques. For example, direct labeling incorporates fluorescent dyes directly into the targets that hybridize to the microarray associated probes (e.g., dyes are incorporated into targets by enzymatic synthesis in the presence of labeled nucleotides or PCR primers). Direct labeling schemes yield strong hybridization signals, typically using families of fluorescent dyes with similar chemical structures and characteristics, and are simple to implement. In preferred embodiments comprising direct labeling of nucleic acid targets, cyanine or Alexa analogs are utilized in multiple-fluor comparative microarray analyses. In other embodiments, indirect labeling schemes are utilized to incorporate epitopes into the nucleic acid targets either prior to or after hybridization to the microarray probes. One or more staining procedures and reagents are used to label the hybridized complex (e.g., a fluorescent molecule that binds to the epitopes, thereby providing a fluorescent signal by virtue of the conjugation of dye molecule to the epitope of the hybridized species). In particular embodiments, a biotin epitope and a fluorescent streptavidin-phycoerythrin conjugate are contemplated.

The present invention is not limited by the nature of the label chosen, including, but not limited to, labels that comprise a dye, fluorescein moiety, a biotin moiety, luminogenic, fluorogenic, phosphorescent, or fluors in combination with moieties that can suppress emission by fluorescence resonance energy transfer (FRET). Further, the probe oligonucleotide and particularly the target sequences may contain positively charged adducts (e.g., the Cy3 and Cy5 dyes, and the like). The oligonucleotides may be labeled with different labels (e.g., one or more probe oligonucleotides may each bear a different label).

The present invention contemplates detecting similar sequences from distinct biological samples using a single microarray hybridization step. Target material (e.g., nucleic acids or polypeptides) within the distinct biological samples may be differently labeled. For example, targets within distinct samples may incorporate different dyes or fluorophores. When differently labeled in one of these ways, the contribution of each specific target sequence to hybridization at a particular probe site can be distinguished. This labeling scheme has several applications. In gene expression studies, for example, the relative rates (i.e., differential-expression) of transcription of one or more particular sequences within a sample can be measured.

Additionally, in particularly preferred embodiments, the detection capabilities of the present invention can be used for detecting the quantities of different versions of a gene within a mixture. Different genes in a mixture to be detected and quantified may be wild type and mutant genes (e.g., as may be found in a tumor sample, such as a biopsy) or different genetic variants of microorganisms. In this embodiment, one might design two sets of one or more probes to be complementary to characteristic sequences in one region of the genome, but one probe set to match the wild-type sequence and one probe set to match the mutant. Quantitative detection of the fluorescence from a microarray reaction performed for a set amount of time will reveal the ratio of the two genes in the mixture. Such analysis may also be performed on unrelated genes in a mixture. This type of analysis is not intended to be limited to two genes. Many variants within a mixture may be similarly measured.

The specificity of the detection reaction is influenced by the aggregate length of the nucleic acid sequences involved in the hybridization of the complete set of the detection (probe) oligonucleotides.

Exemplary fluorophores suitable for labeling microarray samples include those described in Table 2 of “Microarray Biochip Technology” Schena et al., Eaton Publishing 2000.

TABLE 2

| | Aprox. | | | |
|---|---|---|---|---|
| | Absorbance | Aprox. | Structural | |
| Fluorophore | (nm) | Emission(nm) | Partner | Comments |
| FITC¹ | 494 | 518 | | 5-FAM |
| | | | | derivative used |
| | | | | for DNA |
| | | | | sequencing |
| Flour X | 494 | 520 | | Less bright than |
| | | | | FITC |
| Alexa 488 | 495 | 520 | Alexa 432, 546, | |
| | | | 568, and 594 | |
| Oregon Green | 496 | 524 | | |
| 488 | | | | |
| JOE | 522 | 550 | | 6-JOE used for |
| | | | | DNA |
| | | | | sequencing |
| Alexa 532 | 531 | 554 | Alexa 488, 546, | |
| | | | 568, and 594 | |
| Cy3 | 550 | 570 | Cy2, −3.5, −5, | |
| | | | and −5.5 | |
| Alexa 546 | 556 | | Alexa 488, 532, | |
| | | | 568, and 594 | |
| TMR² | 555 | 580 | | 6-TAMRA |
| | | | | used for DNA |
| | | | | sequencing |
| Alexa 568 | 578 | 603 | | Alexa 488, 532, |
| | | | | 546, and 594 |
| ROX³ | 580 | 605 | | 6-ROX used for |
| | | | | DNA |
| | | | | sequencing |
| Alexa 594 | 590 | 617 | | Alexa 488, 532, |
| | | | | 546, and 568 |
| Texas Red | 595 | 615 | | |
| Bodipy 630/650 | 625 | 640 | Bodipy series | |
| Cy5 | 649 | 670 | Cy2, −3, −3.5, | Less soluble in |
| | | | and −5.5 | aqueous than |
| | | | | Cy3 |

¹fluorescein isothiocynate
²tetramethylrhodamine
³X-rhodamine

IV. Approaches to Generating Oligonucleotide Primer Pairs

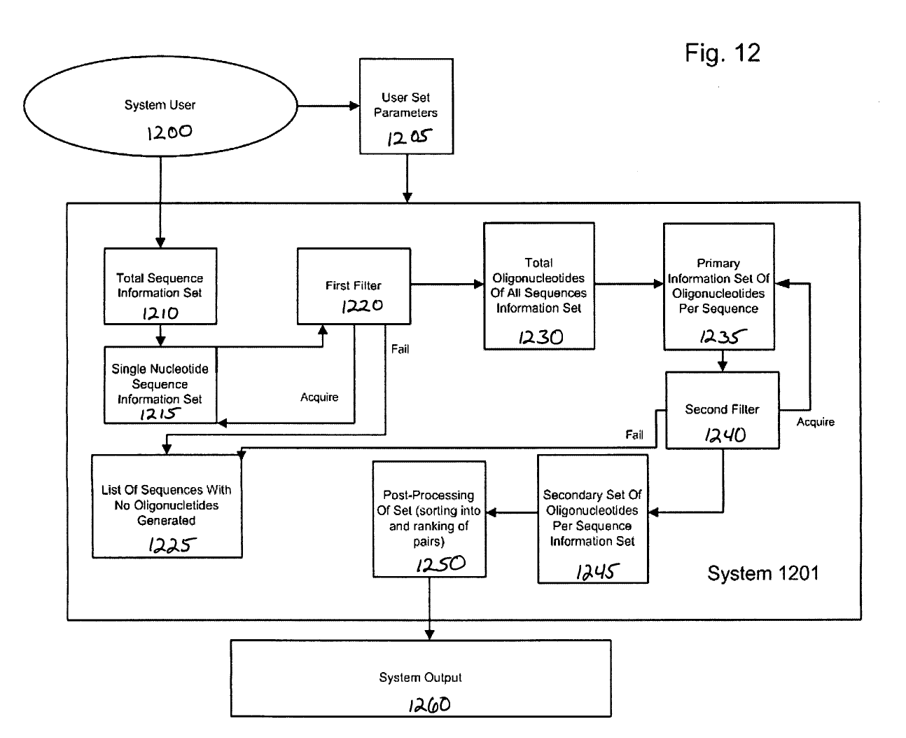

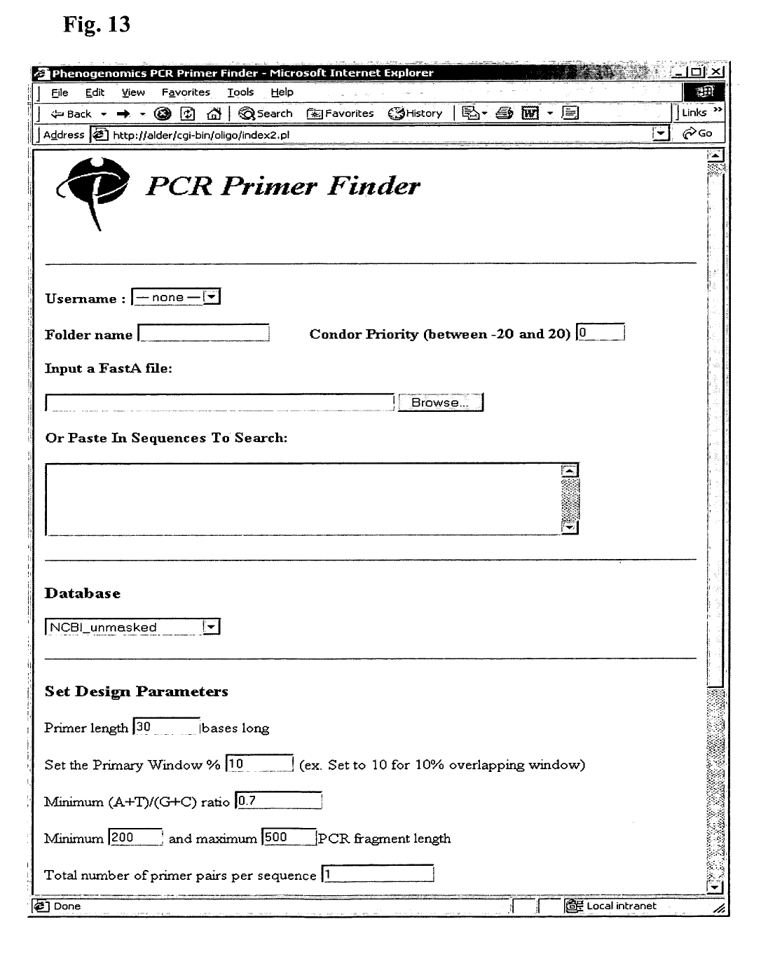

In additional embodiments, the present invention is configured to generate short (e.g., 5 . . . 10 . . . 20 . . . 50 or more) nucleotide sequence pairs suitable for use as primers in nucleic acid sequences, hybridization, and amplification reactions. In particularly preferred embodiments, these short nucleotide sequences are designed as primers for use in polymerase chain reactions (PCR). In some embodiments, the systems and methods of the present invention are configured to generate either or both nucleotide pairs (e.g., PCR primers) and oligonucleotide probe candidates in a particular session. The general architecture and operation of the invention when used to generate nucleotide sequence pairs is similar to that when the invention is used to generate oligonucleotide probe candidates, however, there are a few modifications to accommodate the different output. For example, FIG. 12 shows that system elements 1200-1260 are substantially the same as those described in reference to the exemplary system shown in FIG. 11. A notable exception, system element 1250, is described below. FIG. 13 shows one contemplated user GUI configured for systems configured for generating oligonucleotide primer pairs. In preferred embodiments, the GUI shown in FIG. 13 is substantially similar in system design and operation as that shown in FIG. 2.

A. The Algorithm for Generating Probe Sequences

PCR primer pairs comprise two oligonucleotides, one forward (i.e., 5′ to 3′) and one reverse (i.e., 3′ to 5′). In preferred embodiments of the systems of the present invention, as used to generate primer pairs, the system first generates a list of probe sequences. The probe sequences are subsequently tested for uniqueness using the BLAST algorithm as described above. The resulting unique probes are then evaluated to identify the best pairings between forward and reverse oligonucleotides.

The list of probe sequences is generated in much the same way as described above, by the system moving along within ‘windows’ of about 1000 base pairs along sequences of interest. Repeats and palindromes are screened out (as described above), and the (A+T)/(G+C) ratio is checked. Preferably, the user can input a range of acceptable (A+T)/(G+C) ratios. The user can also input a desired maximum distance between forward and reverse primer patterns. Within each 1 kb window, primers are first sorted according to how close the ratio is to 1, and then sorted (secondarily) by distance from 3′ end of the sequence of interest. In preferred embodiments, only the primers for which there is potential pairing are kept (e.g., user specification dictates that primers must be between 200 and 500 bp apart and there is only one potential primer with adequate complexity and (A+T)/(G+C) ratio within the window, the system discards the primer prior to running the BLAST algorithm since the potential primer would not have another primer with which to pair).

Preferably, the sequences are then checked for uniqueness. This is done basically in the same way as described above using the BLASTn program and then selecting and saving only those probes for which there is only one hit on the genome. In some embodiments there is no timer feature, as all probes should be checked for uniqueness in order to find potential matches. In preferred embodiments, each primer is checked for uniqueness only once, even though each primer might potentially form a pair with several other primers.

Finally, each unique match is checked to see whether it can be paired with another unique probe within an acceptable distance. The one closest to the 3′ end of the sequence of interest is then taken as its reverse complement, making one reverse and one forward primer. The primer pairs are sorted according to similarity of the (A+T)/(G+C) ratio between the two probes, closeness of that ratio to 1, and proximity to the 3′ end of the sequence of interest (See, FIG. 12 at 1250).

There are a number of ways in which the primer pairs may be arranged. In our one embodiment, the ‘best’ pairs according to (A+T)/(G+C) ratio and location near 3′ end, are selected, except no probe sequence is allowed to appear in more than one paring unless there is no other pair available (e.g., given probes 1, 2, 3, and 4 all with the same (A+T)/(G+C) ratio, within acceptable distance from one another, and listed in order of distance from 3′, the system outputs pairs 1-2 and 3-4, even though 1-2 and 1-3 would be the closest two pairs to the 3′ end). One reason for the operation is that it generates more total unique primers than could be used in a hybridization experiment. There are more total options for pairings among four primers than among three. In another embodiment the best possible pairs are reported (e.g., via a GUI), and the user through a GUI is able to manually manipulate or re-order the pairs selection.

Certain of these embodiments, are configured to create PCR primers to verify oligonucleotide results. For this reason, in some embodiments, the system provides a feature for designing PCR primer pairs that surrounds a known oligonucleotide primer site. In this case, the oligonucleotides are stored in a database in a computer readable medium and the PCR system interfaces with the database and determines the location of the oligonucleotide primer. In designing and selecting primer pairs, the system first requires that the primers surround the oligonucleotide site (if possible), and then sorts primer pairs according to (A+T)/(G+C) ratio and distance from the 3′ of the sequence of interest. If there is no suitable surrounding pair, the system selects the pair nearest the oligonucleotide site.

B. The Graphical Output

The informational output of the systems configured (instructed) to generate primer pairs is in much the same format that of systems configured to generate oligonucleotide probe candidates. As mentioned above, in some embodiments, the systems and methods of the present invention are capable of generating both oligonucleotide probe candidates and primer pairs in a single session.

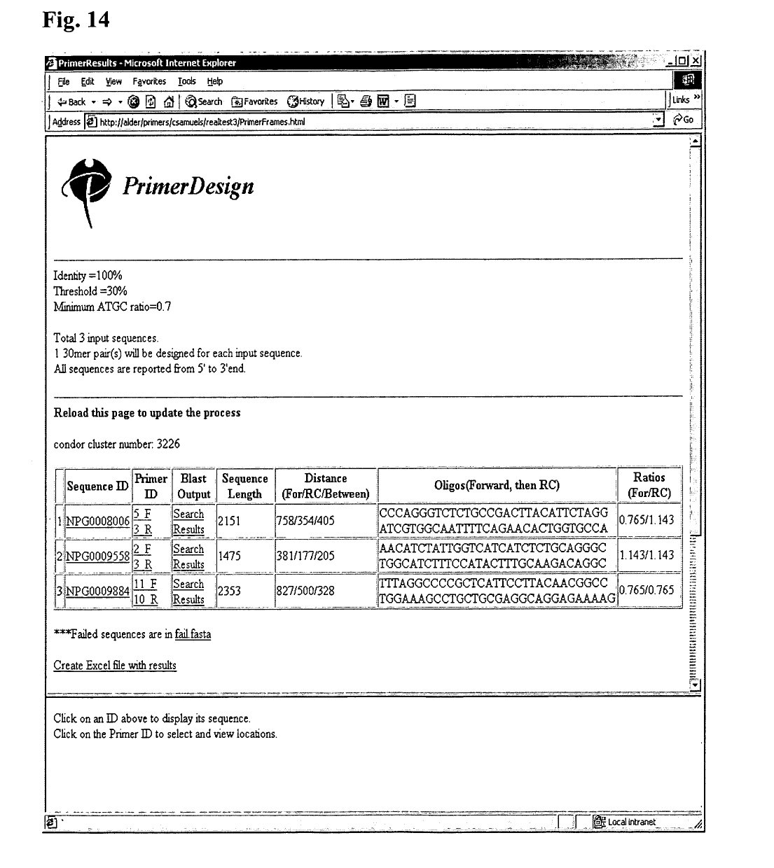

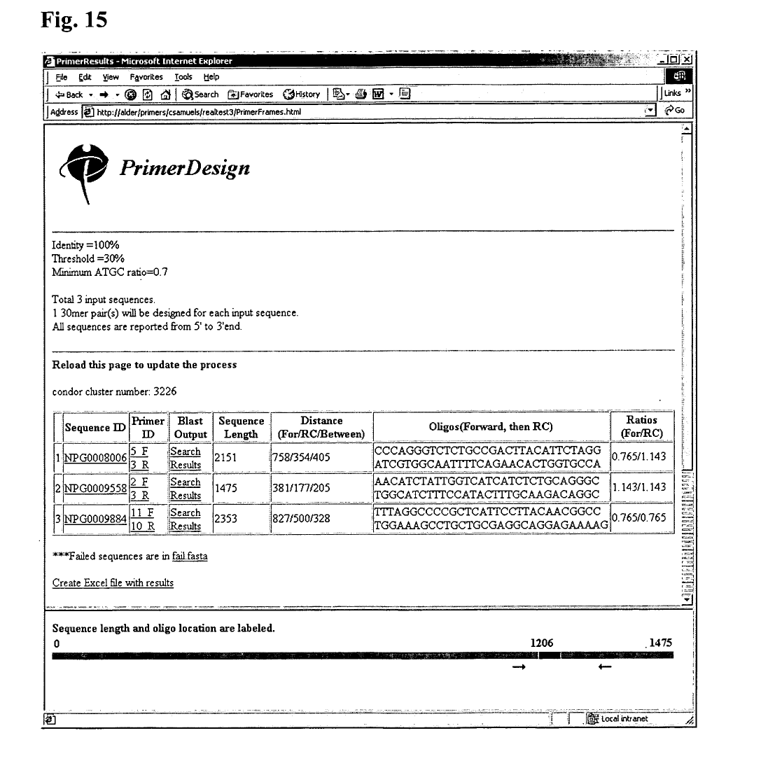

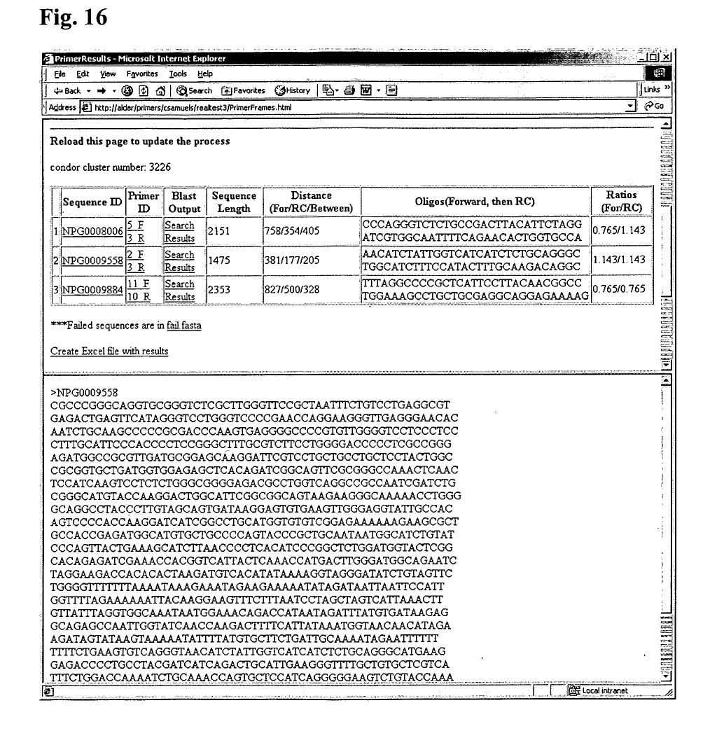

In preferred embodiments, the primer sequences are reported in pairs. (See, FIGS. 14-16). In additional preferred embodiments, the graphical output is in frames, and by clicking on the probe ID in the main output window, the user can view a graphical, colored representation of the location of the PCR and oligonucleotide (if in the database) on the sequence. (See, FIG. 15). Preferably, the colored bars are accurately scaled to represent the length and location of the primers on the entire sequence. As shown in FIG. 15, the oligonucleotide is represented as a colored bar within the sequence, and the PCR primers are shown as arrows below the sequence. In still other embodiments, the GUI is configured such that the user can click and view more than one primer pair at a time. In still further embodiments, the GUI is interactive with the selection of pairs, so that the user can view several primer pairs at a time and by clicking on the arrow representing any primer, select it for inclusion in a list of selected primers. Additionally, when it is selected for a pairing, the system intelligently determines whether the selected primer should be in the forward or reverse direction in that pairing and adjust the output accordingly. By clicking on the sequence ID, the user can view the sequence information in the lower window. By clicking on the blast output link, the user can view the raw blast output for that probe in the lower window. In preferred embodiments, the systems provide a GUI showing sequence information. (See, FIG. 16).

V. Approaches to Preparing Samples for Gene Expression Analysis

The present invention contemplates a variety of distinct biological samples (i.e., targets) are suitable for differential assay against oligonucleotide sequences (i.e., probes) generated by the systems and programs of the present invention. In preferred embodiments of the invention, distinct biological samples are selected to represent contrasting physiological states (e.g., differential expression, etc.), or pathological states (e.g., cancerous versus non-cancerous tissue, infected with pathogen versus non-infected cells, etc.).

The present invention contemplates, but is not limited to, distinct biological samples comprising animal cells and tissue biopsies, bodily fluids (e.g., urine, semen, blood and blood products, saliva, lachrymal secretions, and the like), and solids (e.g., stool, etc.). In particularly preferred embodiments, nucleic acids (e.g., DNA and/or RNA) or polypeptides are isolated from the distinct biological samples (e.g., cells, tissues, bodily fluids, etc.) and subsequently contacted to an array of oligonucleotide sequences designed using the systems described herein.

In some preferred embodiments, nucleic acids isolated from distinct biological samples comprise DNA (e.g., gDNA, or cDNA). In other preferred embodiments, nucleic acids isolated from distinct biological samples comprise RNA (e.g., rRNA, mRNA). In particularly preferred embodiments, mRNA molecules are obtained from the biological sample. The present invention is not limited by the nature or length of the nucleic acids sequences isolated from a particular biological sample. For example, the present invention contemplates employing isolated nucleic acids sequences that encode whole proteins, portions of proteins, or polypeptides.

In still further embodiments, proteins or polypeptides are obtained (and in some cases substantially purified) from distinct biological samples.

Various methods and kits are available for isolating high quality RNA from biological samples. In general, RNA is prepared from, for example, tumor sample by disruption of the tissue in guanidinium isothiocyanate with a tissue cell disrupter (e.g., a Polytron cell disrupter, Brinkmann Instruments, Inc., Westbury, N.Y.) followed by acid phenol extraction and ethanol precipitation. (See e.g., P. Chomczynski and N. Sacchi, Anal. Biochem, 162:156-159 [1987]). In some embodiments, the integrity and amount of RNA obtained from a biological sample is checked by gel electrophoresis using any of a number of known protocols. In some embodiments, mRNA from a sample is isolated from total RNA by the art known oligo(dT) cellulose technique and subsequently labeled cDNA samples will be generated and used for detecting hybridization to arrayed oligonucleotide probe sequences. The present invention is not limited, however, to using mRNA derived cDNA targets for detecting hybridization.

In preferred embodiments, differential expression levels are assayed by contacting RNA molecules obtained from one or more distinct biological samples to a microarray comprising a plurality of oligonucleotide sequences designed by the systems and programs of the present invention. In certain of these embodiments, the RNA molecules are labeled with one or more reporter molecules (e.g., one or more fluorescent dyes, radioisotopes, etc.). Various fluorescent dyes are suitable for use as reporter molecules in the methods of the present invention, including, but not limited to, Cy3 and Cy5. In some embodiments comprising nucleic acids labeled with fluorescent reporter molecules, the reporter molecules are attached (covalently or noncovalently) to one or more of the nucleotide bases comprising the nucleic acid sequence (e.g., Cy3-dUTP and Cy5-dUTP) isolated from the biological sample of interest. In this regard, detailed protocols for labeling nucleic acids with fluorescent probe molecules are well known in the art and readily available at several Web pages, for example, http://brownlab.stanford.edu/protocols.html; National Institute of Environmental Health Sciences (http://dir.niehs.nih.gov/microarray/methods.htm); and the National Human Genome Research Institute (http://www.nhgri.nih.gov/DIR/LCG/15K/HTML/protocol.html). In preferred embodiments, labeled probes are hybridized to microarrays under conditions sufficient to promote probe hybridization to the arrayed oligonucleotide sequences.

The present invention contemplates that any one or more of a number of commercially available computer programs can be used for acquiring data from hybridized microarrays, for example, but not limited to, the IMAGENE (BioDiscovery, Inc., Marina Del Rey, Calif.), and the GENESPRING (Silicon Genetics, Redwood City, Calif.) programs.

In some embodiments, the invention contemplates genes with expression level differences greater than two fold as being differentially expressed. In preferred embodiments, the putative differentially expressed genes are further screened against additional samples of interest from similar pathological conditions. In particularly preferred embodiments, genes that exhibit differential expression in 2 or more different samples of interest are selected for further analysis and for producing antibodies against. The present invention contemplates additional hybridization screening (e.g., ELISA) and display steps provide greater confidence when identifying potential therapeutic targets.

VI. Approaches to Antibody Generation and Humanization

The present invention further contemplates methods for validating the differential expression profiles of one or more genes at the protein level as well as to determine the location of such proteins in various cells and tissues.

Accordingly, in some embodiments, the methods of the present invention further comprise generating peptide antibodies for validating differential gene expression. Briefly, potential therapeutic targets (e.g., cell membrane proteins) are identified based on the differential expression profiles in one or more samples. In some embodiments, the nucleic acid sequence encoding the potential therapeutic target is isolated and cloned into an expression vector, transfected into a proper host, and then expressed. The potential therapeutic targets (e.g., protein antigens) are purified (or partially purified) and subsequently used to immunize an animal that in turn produces antibodies to the target protein. In particularly preferred embodiments, the antibodies generated by the animal are further screened against the original target protein for therapeutic effect using one or more antibody based assays commonly known in the art (e.g., immunohistochemistry or ELISA).

In still further embodiments of the present invention, peptide antibodies are generated as a relatively inexpensive alternative to cloning full-length gene sequences encoding potential therapeutic target proteins into expression vectors for the ultimate production of antibodies. Briefly, most naturally occurring proteins in aqueous solutions have their hydrophilic residues on the surface and their hydrophobic residues buried in the protein interior. Antibodies generally can only bind to epitopes on the surface of proteins. Therefore, the nucleotide sequences selected for generating peptide antibodies preferably encode hydrophilic, surface-oriented residues in the therapeutic target protein. The present invention contemplates using one or more of commercially available software programs (e.g., MACVECTOR, Oxford Molecular Group, Oxford, England; or DNASTAR, DNAStar, Inc., Madison, Wis.) for predicting the target protein's properties and likely secondary structures, such as, hydrophilicity/hydrophobicity, alpha-helix, beta-sheet, and the like. The results of multiple prediction algorithms are used to select highly antigenic regions. In preferred embodiments, the present invention contemplates providing more than one peptide sequence selected to represent a potential therapeutic target for further screening and therapeutic antibody development.

In other preferred embodiments of the present invention, standard immunohistochemistry protocols are followed to validate the expression level of selected putative disease targets at the protein level. This also provides confirmation of i) the cell surface location of the putative targets, and ii) the differential expression.

The present invention contemplates that antibodies (e.g., polyclonal or monoclonal) directed against differentially expressed cell membrane proteins provide useful diagnostic and therapeutic tools in the identification and potential treatment of diseases. In some embodiments, antibodies are generated using the product of an expression vector as an antigen. In some other embodiments, synthetic polypeptides are derived from the amino acid sequences of selected cell membrane proteins that are then used as antigens to immunize an antibody producing animals (e.g., horses, sheep, goats, rabbits, mice, and the like) or cell lines.

In preferred embodiments, polypeptides used to induce specific antibodies comprise at least about 5 amino acids, and preferably at least about 10 amino acids. The present invention contemplates various host animals are suitable for producing therapeutic antibodies, including, but not limited to, horses, sheep, goats, chickens, rabbits, rats, mice, etc. In some embodiments, antibody producing animals are immunized by injection with peptides selected to retain antigenicity. Depending on the host species, various adjuvants, such as complete Freund's or incomplete Freund's adjuvant, aluminum hydroxide, and lysolecithin, are contemplated to increase immunological response in the selected antibody producing animal.

In particularly preferred embodiments, monoclonal antibodies against selected differentially expressed cell membrane proteins are prepared by injecting mice (or rats) with peptides, removing the spleen to obtain B-lymphocytes, fusing the B-lymphocytes with myeloma cells to produce hybridomas, cloning the hybridomas, selecting positive clones that produce antibodies against the antigen (peptides), culturing the clones that produce antibodies, and isolating the antibodies from the hybridoma cultures. The art is familiar with the above-mentioned techniques for producing antibodies from hybridoma cultures.

In other preferred embodiments, antibodies are obtained by in vitro screening of a combinational immunoglobulin library (phage display library).

The present invention further contemplates, methods and compositions of providing antibody-based therapeutics. In particularly preferred embodiments, antibody-based therapeutic compositions further comprise humanized antibodies. In some embodiments, humanized monoclonal antibodies are produced by transferring mouse complementary determining regions from both heavy and light variable chains of a mouse immunoglobulin molecule into a human variable domain. Typically, residues from human antibodies are substituted into the framework region of their murine counterparts. The present invention also contemplates that use of antibody components derived from humanized monoclonal antibodies obviates potential problems associated with the immunogenicity of antibodies comprising murine constant regions. The art well knows methods and techniques for humanizing non-human or partially non-human antibodies. (See e.g., Rader et al., J. Biol. Chem., 275(5):13668-13676 [2000]; Carter et al., Proc. Nat. Acad. Sci. USA, 89:4285-4289 [1992]; herein incorporated by reference in their entireties).

In some other embodiments, human monoclonal antibodies are obtained from transgenic mice that have been engineered to produce specific human antibodies in response to an antigenic challenge. Briefly, in this technique, elements of the human heavy and light chain loci are introduced into mice derived from embryonic stem cell lines that contain targeted disruptions of the endogenous heavy chain and light chain loci. In some embodiments, the invention contemplates transgenic mice that produce human antibodies specific for human antigens, and mice that can be used to produce human antibody-screening hybridomas. (See e.g., Green et al., Nature Genet., 7:13 [1994]; herein incorporated by reference in its entirety).

VII. Verification of Antibody Binding Affinity in Animal Models

The present invention further contemplates using animal models to test the specificity of antibodies to cell membrane proteins identified as being potential mediators of various diseases (e.g., cancer) based on their differential expression in diseased and undiseased biological samples. In some embodiments, animal models provide preliminary information regarding potential toxicity to normal tissues and indications on the effectiveness of the antibody therapies in specific diseased tissues.

One embodiment of the present invention contemplates introducing human tumor tissues or cell lines into model organisms such as mouse. After the tumors have established themselves in the animal, the animal is injected with peptide derived antibodies and tumor growth and the physiological conditions of the animal are monitored.

Antibodies generated according to the teachings of the present invention that are labeled (e.g., antibodies comprising a detectable label such as a dye, fluorophore, chromophore, etc.) can be used in this manner to “image” tumor tissue (as distinct from normal tissue) both in animal models and in humans. Such imaging can be used to design appropriate therapies. Alternatively, antibodies of the present invention are conjugated to toxins so as to deliver a toxin specifically to a cell (e.g., deliver a toxin to a cancer cell or pathogen).

VIII. Pharmaceutical Compositions

Recent advancements in antibody production, stability, humanization, and delivery methods suggested that humanized antibodies can effectively be used as therapeutic agents. One advantage of antibody based pharmaceutical compositions over chemical compounds is their specificity toward target proteins. Additionally, the high specificity of antibodies against their protein targets decreases the possibility of toxicity and adverse side effects as compared to chemical based therapeutics.

The present invention provides pharmaceutical compositions that comprise one or more antibody species, alone or in combination with one or more other agents (e.g., a stabilizing compound) that are administered in any sterile, biocompatible pharmaceutical carrier, including, but not limited to, saline, buffered saline, dextrose, and water.

In preferred embodiments, the pharmaceutical compositions comprise one or more species of humanized antibodies. Those skilled in the art are familiar with methods of humanizing antibodies.

The compositions and methods of the present invention find use in treating diseases or in altering physiological states characterized by the differential expression of genes encoding atypical cell membrane proteins. Therapeutic antibody compositions can be administered to the patient intravenously in any pharmaceutically acceptable carrier such as physiological saline. Standard methods for intracellular delivery of peptides can be used (e.g., delivery via liposome or dendrimers). Such methods are well known to those of ordinary skill in the art. In preferred embodiments, the formulations of this invention are useful for parenteral administration, such as intravenous, subcutaneous, intramuscular, and intraperitoneal.

As is well known in the medical arts, dosage levels for any one patient depend upon many factors, including the patient's size, body surface area, age, the particular compound to be administered, sex, time and route of administration, general health, and interaction with other drugs being administered.

Therapeutic administration of a polypeptide intracellularly can also be accomplished using gene therapy techniques. Accordingly, in some embodiments of the present invention, therapeutic antibodies can be administered to a patient alone, or in combination with other nucleotide sequences, drugs or hormones or in pharmaceutical compositions where it is mixed with excipient(s) or other pharmaceutically acceptable carriers.

Depending on the condition being treated, the pharmaceutical compositions of the present invention may be formulated and administered systemically or locally. Techniques for formulation and administration may be found in the latest edition of “Remington's Pharmaceutical Sciences” (Mack Publishing Co., Easton Pa.). Suitable routes may, for example, include oral or transmucosal administration; as well as parenteral delivery, including intramuscular, subcutaneous, intramedullary, intrathecal, intraventricular, intravenous, intraperitoneal, or intranasal administration.

For injection, the pharmaceutical compositions of the invention may be formulated in aqueous solutions, preferably in physiologically compatible buffers such as Hanks' solution, Ringer's solution, or physiologically buffered saline. For tissue or cellular administration, penetrants appropriate to the particular barrier to be permeated are used in the formulation. Such penetrants are generally known in the art.

In other embodiments, the pharmaceutical compositions of the present invention can be formulated using pharmaceutically acceptable carriers well known in the art in dosages suitable for oral administration. Such carriers enable the pharmaceutical compositions to be formulated as tablets, pills, capsules, liquids, gels, syrups, slurries, suspensions and the like, for oral or nasal ingestion by a patient to be treated.

Pharmaceutical compositions suitable for use in the present invention include compositions wherein the active ingredients are contained in an effective amount to achieve the intended purpose. For example, an effective amount antibody may be that amount that suppresses cell surface signal transduction. Determination of effective amounts is well within the capability of those skilled in the art, especially in light of the disclosure provided herein.

In addition to the active ingredients these pharmaceutical compositions may contain suitable pharmaceutically acceptable carriers comprising excipients and auxiliaries that facilitate processing of the active compounds into preparations that can be used pharmaceutically. The preparations formulated for oral administration may be in the form of tablets, dragees, capsules, or solutions.

The pharmaceutical compositions of the present invention may be manufactured in a manner that is itself known (e.g., by means of conventional mixing, dissolving, granulating, dragee-making, levigating, emulsifying, encapsulating, entrapping or lyophilizing processes).

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

For any compound or antibody used in the methods of the present invention, the therapeutically effective dose can be estimated initially from cell culture assays. Then, preferably, dosage can be formulated in animal models (particularly murine models) to achieve a desirable circulating concentration range of antibodies.

A therapeutically effective dose refers to that amount of a particular antibody that ameliorates symptoms of the disease state. Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index, and it can be expressed as the ratio LD50/ED50. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors that may be taken into account include the severity of the disease state; age, weight, and gender of the patient; diet, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation. Normal dosage amounts may vary from 0.1 to 100,000 micrograms, up to a total dose of about 1 g, depending upon the route of administration.

Experimental

The following examples are provided in order to demonstrate and further illustrate certain preferred embodiments and aspects of the present invention and are not to be construed as limiting the scope thereof.

In the experimental disclosure that follows, the following abbreviations apply: N (normal); M (molar); mM (millimolar); mM (micromolar); mol (moles); mmol (millimoles); mmol (micromoles); nmol (nanomoles); pmol (picomoles); g (grams); mg (milligrams); mg (micrograms); ng (nanograms); l or L (liters); ml (milliliters); ml (microliters); cm (centimeters); mm (millimeters); mm (micrometers); nm (nanometers); and ° C. (degrees Centigrade).

### EXAMPLE 1

### Prediction of Transmembrane Spanning Protein Sequences

In this example, protein sequence databases were screened for sequences that are predicted to encode proteins with one or more likely transmembrane domains.

Briefly, the following protein sequence databases: (e.g., the proteome database (http://www.ebi.ac.uk/proteome/); the ensemble predicted unconfirmed protein database [http://www.ensembl.org/]; and GenBank non-redundant [nr] protein database, [http://www.ncbi.nlm.nih.gov/Genbank/index.html] limited by key word search to human sequences) were screened for potential cell membrane proteins by analysis with the maxH (See, Boyd et al., Protein Sci., 7(1):201-205 [1998]); and TMHMM (See, Krogh et. al., J. Mol. Biol., 305:567-580 [2001]) transmembrane domain predicting software programs. The Perl script New_maxH_v3.3.pl was downloaded from http://beck2.med.harvard.edu and run locally on a Redhat v. 7.0 Linux system. Protein sequences with maxH p-values >0.68 were selected as candidates likely to contain one or more helical transmembrane domains. The software package TMHMM was licensed from Technical university of Denmark (Lyngby, Denmark) and run locally on a Sun Microsystems Blade100 computer (Sun Microsystems, Inc., Palo Alto). Sequences with one or more TMHMM predicted transmembrane domains were selected as membrane protein candidate. The results from two different programs show significant overlap as expected. However, the TMHMM program generates more potential membrane protein candidates (See, Table 1).

A total of 282,776 individual protein sequences were assembled in a local sequence database from the above mentioned protein databases. In particular, the local sequence database comprised about 30,585 sequences from the Proteome database, about 183,706 from the Ensembl Unconfirmed Database, and about 65,485 sequences from the NCBI nr database, respectively.

### EXAMPLE 2

### Prediction of Nucleotide Sequences Encoding Transmembrane Spanning Proteins

In this example, the GENSCAN predicting program (licensed from Stanford University) was used to screen the GenBank genomic database to identify nucleic acid sequences that potentially encode cellular proteins. The nucleic acid sequences identified were then passed through the transmembrane domain prediction programs to identify putative membrane proteins as described in Example 1.

### EXAMPLE 3

### Generation of Oligonucleotide Sequence

In this example, the nucleic acid sequences identified as potentially encoding cell membrane spanning proteins are synthesized using standard phosphoramidite chemistry by standard column based oligonucleotide synthesizer. Briefly, oligonucleotides representing the sequences of interest, such as, sequences that encode putative transmembrane domains (motifs) are selected using the systems and programs described above. The selection of oligonucleotides are based on several key factors that include, but are not limited to, the (A+T)/(G+C) ratio, the location, and the uniqueness of the sequence. The unique oligonucleotide sequences were then sent to an oligosynthesizer. The synthesized oligonucleotides were then dried and resuspended in 3×SSC.

### EXAMPLE 4

### Production of Microarrays

This example describes the arraying of the oligonucleotide sequences generated in Example 3 onto a solid microarray substrate.

Briefly, glass slides were washed with 2.5M of sodium hydrochloride solution containing 60 of ethanol (100 g of NaOH dissolved in 400 ml of water then added 600 ml of EtOH) to remove oils and debris that could prevent the coating of poly-L-lysine. Quickly transfer the slides to clean water to remove the NaOH. The clean slide were then dip into the Poly-L-Lysine solution (70 ml Poly-L-Lysine mixed with 70 ml of PBS then added 560 ml of water to the solution) for one hour. Poly-L-lysine is coated onto the glass slides to increase the binding efficiency of oligonucleotides. The coated slides were then washed with water and dried in oven before use. Oligonucleotides generated by the systems and programs of the present invention (described infra) are suspended in 3×SSC with a concentration of 50 mM. A microarray printer (e.g., Qarray from Genetix Pharmaceuticals, Inc., Cambridge, Mass.; or the GMS 418 from Affymetrix, Inc., Santa Clara, Calif.) is used to array the oligonucleotides on poly-L-lysine coated glass slides. The arrayed nucleotide sequences are fixed on the slide by applying the slides with 50 mJ of UV light. To block unused DNA binding site, the sides were placed in succinic anhydride solution (2.8 g succinic anhydride in approx. 150 mL 1-methyl-2-pyrrolidi and 7 mL sodium borate). The slides are ready to use in hybridization experiment after rinse with water.

### EXAMPLE 5

### Isolating RNA from Biological Samples of Interest

This example describes the isolation of RNA from tumor sample using the Qiagen (Qiagen, Inc., Valencia, Calif.) as per the manufactures instructions.

Briefly, fresh or frozen tissues were homogenized in lysis buffer provided in the RNA isolation kit. Transfer of the tissue lysate to RNA column discard the flow-through. Wash the column with washing buffer provided in the kit to remove unwanted cellular materials. The purified RNA was then eluted from the column with elution buffer from the kit. The RNA were then precipitated with ethanol and redissolved in RNAse-free water with desire concentration (about 5 microgram/microliter). RNA samples were stored at −80° C. for long-term storage.

### EXAMPLE 6

### Target Labeling

In this example, RNA isolated from biological samples of interest, as described in Example 5, were labeled with fluorescent dyes and attached to a microarray.

About 30 microgram of total RNA were mixed with 5 microgram of oligo dT in a 1.5 ml RNAse free micorfuge tube. To anneal the oligo dT primer with RNA, the tube with RNA and oligodT was incubated at 70° C. for 10 minute and then place on ice for 2 minute. Added reverse transcriptase buffer, DTT, 400 units of Superscript II reverse transcriptase, dNTP to the tube at a final concentration of 12.5 mM, and Cy3 or Cy5 labeled dUTP at a final concentration of 1 mM. The tube was then incubated in 42° C. incubator for 1 hour. The cDNA products were coupled with fluorescent dyes.

### EXAMPLE 7

### Hybridization and Post Hybridization Handling Procedures

In this example, the fluorescent labeled cDNA from Example 6 were resuspended in final concentration of 3×SSC, 0.2% SDS, 10 microgram of poly dA oligo and 10 microgram of human Cot-1 DNA (for human gene array, if it is for mouse array, the human Cot-1 will be replaced with mouse Cot-I DNA). Fluorescent labeled cDNAs from the control and diseased RNA samples were then be mixed and denatured by heating at 100° C. for 2 minutes prior added into the hybridization mixture. The microarray slides as described in Example 4 were block with blocking solution to prevent the non-specific binding of the probe. The denatured cDNA mixture were applied onto the slides and covered with cover slip for hybridization at 63° C. for 14-18 hours. After the hybridization, the slides were washed with 1×SSC, 0.03% SDS to remove the cover slip. High stringent wash as described before were used to remove non-specific hybridization signal.

### EXAMPLE 8

### Hybridization Data Acquisition and Analysis

In this example, microarrays from EXAMPLE 7 were scanned with a fluorescent scanner (e.g., Axon Genepix 400a) as soon as the washing procedure was finished to prevent signal loss due to light exposure during the process. The scanned results were analyzed with programs such as Genepix and Genespring to compare the fluorescent signals between the control and diseased samples. The spots with at least 2-fold-signal differences will then be marked and termed as putative differentially expressed spots. The corresponding gene sequences will be selected and grouped in genes that are putatively differentially expressed between the control and diseased samples.

### EXAMPLE 9

### Generation of Polypeptides Representing Fragments of Differentially Expressed Genes

In this example, the differential display results from Example 8 are correlated to the arrayed pattern of oligonucleotides. Thus, the data sets generated are interpreted to show which hybridization markers correspond to differentially expressed cell membrane proteins. Polypeptides are synthesized. After differentially-expressed proteins are identified by the corresponding oligonucleotides, either full length proteins or peptides will be generated or synthesized. To produce full-length proteins, the coding DNA sequences will be cloned into a protein expression vector (e.g., pQE30, Qiagen). The plasmid will be introduced into bacteria. Proteins are synthesized by transformed bacteria under specific stimulation. Bacteria will be harvested and the protein will be purified according to the manufacture instructions (e.g., Qiagen Ni-NTA magnetic agarose beads). Amino acid sequences of the differentially-expressed proteins will be analyzed by programs (e.g., MacVector) to identify the regions which are more antigenic. Peptides of 5 to 20 amino acid residues corresponding to those antigenic regions will be synthesized with a peptide synthesizer.

### EXAMPLE 10

### Generation of Antibodies to Selected Polypeptides

In this example, the polypeptides selected and isolated from the differential display routines described in Example 9 are used to generate antibodies.

Briefly, purified proteins or synthetic peptides will be used to immunize mice to generate antibody-forming cells. Proteins or peptides will be mixed with adjuvant (e.g., ImmunEasy a Mouse Adjuvant from Qiagen) and subcutaneously injected into mouse foot pads. Mice will be boosted at least once and screened for highest antibody titer before the spleens are harvested. Spleen cells will be fused with a myeloma cell line to develop antibody-producing hybridomas. ELISA will be used to determine the titer of the immunized mice and of the antibody generated by each individual hybridoma clone.

### EXAMPLE 11

### Confirming Cell Membrane Protein Expression and Location

Mouse monoclonal antibodies generated in Example 10 will be used to confirm the expression and location of the differentially-expressed proteins by immunohistochemistry. Paraffin-fixed fresh or frozen control and diseased tissues will be sectioned and placed onto glass slides. Mouse monoclonal antibodies from Example 10 will be used to hybridize the sectioned tissues. To detect the hybridization, enzyme-labeled secondary antibody (e.g., peroxidase-labeled goat monoclonal antibodies against mouse immunoglobulin heavy chain, Pierce) will be used according to the manufacture's protocol.

### EXAMPLE 12

### Animal Models

Nude mice will be used to inject human tumor cell lines or primary tumor cells to induced tumor formation. After human tumor is established in the mice, mice will be injected with the monoclonal antibody from Examples 10 and 11. The size of the tumor will be monitored to examine the effect of antibody treatment.

### EXAMPLE 13

### Humanization of Antibodies

Antibody (immunoglobulin) recognizes antigen with its variable regions of the heavy and light chains. To humanize mouse antibody, the variable regions of the mouse antibodies in Example 12 will be transferred to the constant regions of human immunoglobulin. RNA isolated from the antibody-generating hybridoma (Example 10) will be reverse-transcribed to generate cDNA. The variable regions of the mouse immunoglobulin heavy and light chain will be amplified by PCR with specific primers and the amplified products will be ligated to vectors expressing the constant regions of human immunoglobulin heavy and light chains, respectively.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in material science, chemistry, and molecular biology or related fields are intended to be within the scope of the following claims.

1. A method, comprising:
a) providing:
i) RNA from two or more distinct biological samples,
ii) a plurality of nucleic acid sequences immobilized on a solid support, wherein each of said sequences comprise at least one region encoding a hydrophobic domain;
b) contacting said immobilized sequences with said RNA under conditions such that there is hybridization of at least a portion of said RNA with at least a portion of said immobilized sequences; and
c) detecting hybridization.

2. The method of claim 1, wherein said plurality of nucleic acid sequences immobilized on a solid support are arrayed in a grid.

3. The method of claim 1, wherein said RNA from two or more distinct biological samples is selected from the group consisting of total RNA and total mRNA.

4. The method of claim 1, wherein each of said two or more distinct biological samples comprise eukaryotic cells.

5. The method of claim 4, wherein said eukaryotic cells comprise mammalian cells.

6. The method of claim 5, wherein said mammalian cells comprise human cells.

7. The method of claim 6, wherein said human cells are selected from the group consisting of diseased cells and non-diseased cells.

8. The method of claim 7, wherein said diseased cells are cancer cells.

9. The method of claim 6, wherein said human cells differentially express one or more mRNA of interest.

10. The method of claim 9, wherein said one or more mRNA of interest comprises a viral mRNA.

11. The method of claim 1, wherein each of said two or more distinct biological samples comprise prokaryotic cells.

12. The method of claim 11, wherein said prokaryotic cells are selected from the group consisting of pathogenic bacterial cells and non-pathogenic bacterial cells.

13. The method of claim 1, wherein said plurality of nucleic acid sequences comprise DNA.

14. The method of claim 13, wherein said DNA comprises cDNA.

15. The method of claim 14, wherein said nucleic acid sequences encode membrane proteins.

16. The method of claim 15, wherein said membrane proteins comprise one or more transmembrane domains.

17. The method of claim 1, further comprising d) distinguishing between the i) hybridization of RNA from of both said first and second samples to the same immobilized sequence and ii) hybridization of RNA of either said first and second samples to a particular immobilized sequence, so as to identify differentially-expressed genes.

18. The method of claim 17, further comprising step e) comprising the step of generating polypeptides corresponding to at least a portion of said one or more differentially-expressed genes.

19. The method of claim 18, further comprising step f) comprising the step of generating antibodies to said polypeptide corresponding to at least a portion of said one or more differentially-expressed genes.

20. The method of claim 19, further comprising step g) comprising the step of i) contacting said two or more biological samples with said antibodies and ii) detecting the extent of binding of said antibodies to said two or more biological samples.

21. The method of claim 1, wherein said plurality of nucleic acid sequences is selected from a genomic database of nucleic acid sequences.

22. The method of claim 21, wherein said genomic database comprises nucleic acid sequences from eukaryotic cells.

23. The method of claim 22, wherein said eukaryotic cells are human cells.

24. The method of claim 21, wherein said genomic database comprises nucleic acid sequences from prokaryotic cells.

25. The method of claim 21, wherein said genomic database is queried with a system configured for selecting gene sequences encoding membrane proteins and fragments thereof.
